(19) United States
(12) Patent Application Publication
(10) Pub. No.: US 2026/0266814 A1
(43) Pub. Date: Sep. 10, 2026

(54) DEPLETION OF HIGH-ABUNDANCE PLASMA PROTEINS USING IN SITU GROWTH OF METAL-ORGANIC FRAMEWORKS (MOFS) FOR PROTEOMIC ANALYSIS AND BIOMARKER DISCOVERY

(71) Applicant: Wisconsin Alumni Research Foundation, Madison, WI (US)

(72) Inventors: Ying Ge, Madison, WI (US); Song Jin, Madison, WI (US); Emily Reasoner, Madison, WI (US); Hsin-Ju Chan, Madison, WI (US)

(21) Appl. No.: 19/073,721

(22) Filed: Mar. 7, 2025

(51) Int. Cl. G01N 33/543; G01N 33/49; G01N 33/573; G01N 33/68

(52) U.S. Cl. CPC G01N 33/54353; G01N 33/491; G01N 33/573; G01N 33/686; G01N 2333/4716; G01N 2333/70503; G01N 2333/75; G01N 2333/76; G01N 2333/775; G01N 2333/79; G01N 2333/805; G01N 2333/8125

(57) ABSTRACT

Methods and kits for selectively separating biomolecules in complex samples, such as in blood serum, plasma, and other biological fluids. Metal-organic frameworks (MOFs) are formed around selected biomolecules in a mixture during an in-situ growth process, thereby allowing these biomolecules to be separated more easily from molecules that are not encapsulated by the MOFs. One aspect of the invention separates high-abundance proteins from a biological fluid to enhance the detection and characterization of lower-abundance or harder to detect proteins. This allows the lower-abundance or harder to detect proteins to be more easily detected and accurately analyzed, for example, by mass spectrometry. This is particularly useful in blood samples that contain highly-abundant plasma proteins that interfere with the mass spectrometry analysis of lower-abundant proteins and and protein biomarkers.

### STATEMENT REGARDING FEDERALLY SPONSORED RESEARCH OR DEVELOPMENT

This invention was made with government support under GM117058 awarded by the National Institutes of Health. The government has certain rights in the invention.

### BACKGROUND OF THE INVENTION

Detection and characterization of protein biomarkers in plasma is critical to early disease detection and improved patient outcomes.¹⁻³ Liquid chromatography-mass spectrometry (LC-MS) provides the most comprehensive characterization of proteins.⁴⁻⁶ However, the detection of low-abundance protein biomarkers by LC-MS is challenging due to the presence of highly abundant plasma proteins, such as human serum albumin (HSA), which make-up ~99% of the plasma proteome.^{3,7} Thus, plasma proteomics studies often required the depletion of highly abundant proteins using immunoaffinity technologies,⁸⁻¹⁴ anion-exchange chromatography,^{15,16} protein precipitation,¹⁷⁻¹⁹ or synthetic materials.²⁰⁻²⁸ Immunoaffinity-based depletion methods, which are commonly used in blood proteomics and commercially available, utilize antibodies immobilized on columns and spin-columns that specifically bind to high-abundance proteins.⁸⁻¹⁴ Protein precipitation strategies utilize differences in solubility to precipitate highly abundant proteins using reagents such as perchloric acid, followed by solid phase extraction (SPE) of the depleted fraction to remove acid prior to bottom-up proteomics sample preparation.¹⁷⁻¹⁹ Several synthetic materials-based solutions have also been developed for plasma fractionation to expand proteome coverage, including combinatorial peptide ligands immobilized on beads²³⁻²⁶ and multi-nanoparticle protein coronas which have increased the number of protein and proteoforms identified from serum.²⁰⁻²² Although these techniques have demonstrated promise for depletion of abundant proteins and plasma proteomics, they can be costly,^{8-14,20-22} increase sample handling requirements,^{8-14,17,18} limit throughput,^{8-14,29} and/or lack reproducibility.^{17,30}

Metal-organic frameworks (MOFs) are another class of materials that could be explored for proteomics applications. A major benefit to using MOFs for proteomics applications is their simple synthesis, tunability, low precursor cost, biocompatibility, and ultra-high surface area.³¹⁻³⁴ MOFs can strongly interact with proteins and peptides through metal coordination, organic ligand affinity, and post-synthetic modifications. For example, their cage-like, porous structures have been utilized to selectively enrich for phosphorylated or glycosylated peptides.³⁵⁻³⁸ Because MOFs can be readily dissolved using chelating agents or by lowering pH, captured cargo is easily recovered for analysis, whereas other nanoparticle-based strategies often require harsh elution conditions including surfactants that are not MS compatible. Despite these promising progresses in MOF-based proteomics enrichment, there are few studies that utilize the cage-like framework and ultra-high surface area of MOFs to separate intact proteins from complex protein mixtures, as intact protein diameter is typically larger than the pore sizes of traditional MOFs.³⁹

As used herein and in this field, composites that contain molecules encapsulated within a MOF are designated using the “@” symbol (i.e., “protein@MOF composites” or “biomolecule@MOF composites”), although a hyphen may also be used herein (i.e., “protein-MOF composites” or “biomolecule-MOF composites”).

Recent advances show that proteins can be encapsulated within MOFs to impart chemical and physical stability to the proteins in native state, and the formed protein@MOF biocomposites have been exploited for protein and drug delivery, sensing, and catalysis applications.⁴⁰⁻⁵³ Two major ways to encapsulate proteins within MOFs include post-synthetic packaging of proteins in mesoporous MOFs,^{41,44,48,49,53} and bio-mineralization of MOFs in protein solutions.^{46,50-52,54-56} However, synthesizing mesoporous MOFs can be challenging, requiring organic solvents and high temperatures, and must be tuned for each target protein.⁵³ Biomineralization, on the other hand, immobilizes proteins within MOFs through facile in situ synthesis of protein@MOF biocomposites in aqueous protein solutions.^{46,50-52,54-56}

MOFs undergo protein-mediated nucleation in non-denaturing aqueous conditions and grow around and encapsulate intact proteins in their folded conformation.^{55,57-59} Zeolitic imidazolate framework-8 (ZIF-8) is the most widely studied MOF for this purpose due to its ready formation in aqueous conditions.^{60,61} Most reports on the applications of protein@MOF biocomposites only study immobilization of individual proteins from a standard solution, and this MOF growth and protein encapsulation process has not been exploited to separate proteins in a multi-protein mixture. Notably, successful protein encapsulation within ZIF-8 has been shown to be strongly dependent on protein surface chemistry such as charge and Zn-binding capacity,⁶²⁻⁶⁴ thus there is the potential to separate proteins in a complex mixture based on encapsulation efficiency using in situ growth without requiring any front-end materials synthesis prior to protein enrichment.

### BRIEF SUMMARY OF THE INVENTION

The present invention provides methods and kits for selectively separating biomolecules in complex samples, such as in blood serum and other biological fluids. This invention utilizes metal-organic frameworks (MOFs) that form around biomolecules in a mixture, thereby allowing these biomolecules to be separated from molecules that are not encapsulated by the MOFs. In one aspect of the invention, the separated biomolecules and/or the molecules that are not encapsulated by the MOFs are analyzed after being separated, including but not limited to mass spectrometry analysis.

An embodiment of the invention provides a method for separating biomolecules in a mixture comprising the step of encapsulating one or more biomolecules in the mixture within a metal-organic framework (MOF), thereby forming encapsulated biomolecule@MOF composites. Encapsulating the one or more biomolecules within the biomolecule@MOF composites comprises adding a) an organic ligand, and b) metal ions or a metal ion precursor able to form the metal ions, to an aqueous solution containing the mixture, where the organic ligand and metal ions surround each of the one or more biomolecules and form the encapsulated biomolecule@MOF composites. The encapsulated biomolecule@MOF composites in the aqueous solution are then separated from biomolecules that are not encapsulated. Preferably, the one or more biomolecules are proteins or polypeptides.

In an embodiment, a portion of the biomolecules comprise proteins having a post-translation modification (PTM), and the one or more encapsulated proteins are separated based on the degree and types of PTMs. Optionally, the encapsulated proteins are proteins having the PTM and the unencapsulated proteins are proteins that do not have the PTM, proteins having a different type of PTM, and/or proteins having a different amount of the same PTM (i.e., proteins having a higher or lower degree of glycosylation or other PTM). Alternatively, some PTMs reduce or do not permit nucleation and formation of the MOFs and the encapsulated proteins are proteins that do not have the PTM (or have a lower amount of the PTM), which are then separated from unencapsulated proteins having the PTM (or having a greater amount of the PTM). In an embodiment, the PTM is glycosylation and the encapsulated proteins are unmodified proteins that are separated from unencapsulated glycoproteins. In an embodiment, the encapsulated proteins are glycoproteins that are separated from unencapsulated proteins that contain a greater amount (or alternatively, a lower amount) of glycosylation.

After the encapsulated biomolecule@MOF composites are separated from the unencapsulated biomolecules, one or more of the separated encapsulated biomolecule@MOF composites, unencapsulated biomolecules, or both, are analyzed. Suitable analytical techniques include, but are not limited to, mass spectrometry (MS), liquid chromatography (LC), LC-MS, electrophoresis, X-ray crystallography, NMR spectroscopy, Western blotting, small angle x-ray scattering (SAXS), Fourier-transform infrared spectroscopy (FTIR), and quantitative analysis.

Optionally, the MOF material encapsulating the one or more biomolecules is dissolved under mild, and preferably MS-compatible conditions (including conditions where a surfactant is not present), allowing for easy removal and recovery of the biomolecules, potentially retaining the biomolecules (particularly proteins) in their native structural state. In an embodiment, the MOF material is degraded or dissolved using ethylenediamine-tetraacetic acid (EDTA), ethylene glycol-bis(β-aminoethyl ether)-N,N,N′,N′-tetraacetic acid (EGTA), a phosphate buffer, or an acid solution. Preferably, the MOF material is degraded or dissolved using a phosphate buffer in order to preserve the native state of the protein, and to avoid potentially denaturing metal-binding proteins and breaking up protein complexes. In an embodiment, the method further comprises collecting the separated encapsulated biomolecule@MOF composites, degrading the MOF, and recovering the one or more biomolecules.

In an embodiment, the aqueous solution comprises a buffer solution. Suitable buffer solutions include known buffer solutions commonly used in cell culture, biochemical analysis, and other biological studies, including but not limited to HEPES (2-[4-(2-hydroxyethyl)piperazin-1-yl]ethanesulfonic acid), Tris (tris(hydroxymethyl)aminomethane), PBS (Phosphate buffered saline), TAPS ([tris(hydroxymethyl)methylamino]propanesulfonic acid), Bicine (2-(bis(2-hydroxyethyl)amino)acetic acid), Tricine (N-[tris(hydroxymethyl)methyl]glycine), TAPSO (3-[N-tris(hydroxymethyl)methylamino]-2-hydroxypropanesulfonic acid), MOPS (3-(N-morpholino)propanesulfonic acid), PIPEs (piperazine-N,N′-bis(2-ethanesulfonic acid) buffer solutions, and combinations thereof. Preferably, the buffer solution provides a pH to the aqueous solution between between 5.0 and 10.0, more preferably between 6.0 and 9.0, between 6.5 and 8.5, between 7.0 and 8.0, or between 7.25 and 7.5. In an embodiment, MOF growth is performed in a buffer solution where the native structure and function of the one or more encapsulated biomolecules are preserved.

After the encapsulated biomolecule@MOF composites and unencapsulated biomolecules are separated, the buffer solution is optionally removed and replaced with a MS-compatible solution. An MS-compatible solution is a solution able to be injected in a mass spectrometer device without adversely affecting the sample or the MS analysis. In an embodiment involving native MS or native top-down MS analysis, MS-compatible solutions include, but are not limited to, alkylammonium acetate/bicarbonate solutions. In an embodiment, MS-compatible solutions include, but are not limited to, ammonium acetate, ammonium bicarbonate, triethylammonium acetate, triethylammonium bicarbonate, ethylenediammonium diacetate, and combinations thereof. Preferably, the MS-compatible solution has a pH between 5.0 and 10.0, more preferably between 6.0 and 9.0, between 6.5 and 8.5, between 7.0 and 8.0, or between 7.25 and 7.5, and is preferably does not alter the structure of the sample to be analyzed. In an embodiment, the method further comprises replacing the buffer solution with a mass spectrometry (MS)-compatible solution after the encapsulated biomolecule@MOF composites are separated from the aqueous solution, and performing MS analysis on the separated unencapsulated biomolecules, encapsulated biomolecule@MOF composites, or both.

In an embodiment involving denatured protein analysis, including denatured top-down MS, middle-down MS, and bottom-up MS analysis, the unencapsulated biomolecules and/or encapsulated biomolecule@MOF composites are desalted using water containing water-miscible organic solvents, including but not limited to acetonitrile, isopropanol, and ethanol, and optionally one or more acids, including but not limited to formic acid and trifluoroacetic acid, prior to MS analysis.

As used herein, a “biological fluid” refers to a liquid from the body of an organism, including but not limited to blood, cerebrospinal fluid, lymph, saliva, mucus, urine, sweat, synovial fluid, pleural fluid, pericardial fluid, peritoneal fluid, gastric fluid, semen, amniotic fluid, secretions, components thereof, and mixtures thereof. Preferably, biological fluids comprise proteins, cells, metabolites and/or biomolecules contained therein. In an embodiment, the biological fluid is a human biological fluid. Preferably, the biological fluid comprises a whole blood sample, blood plasma sample, or blood serum sample, from a subject.

In an embodiment, the biological fluid comprises a whole blood sample, blood plasma sample, or blood serum sample, and the one or more biomolecules that are encapsulated and separated from the mixture are high-abundant proteins found in blood. For example, in an embodiment, the one or more biomolecules that are encapsulated and separated comprise one or more proteins selected from the group consisting of: albumin, complement C3, serotransferrin, alpha-2-macroglobulin, alpha-1-antitrypsin, apolipoprotein A-I, apolipoprotein A-II, haptoglobin, immunoglobulin heavy constant gamma 1, immunoglobulin heavy constant gamma 2, immunoglobulin heavy constant gamma 3, immunoglobulin heavy constant gamma 4, immunoglobulin heavy constant alpha 1, immunoglobulin heavy constant alpha 2, immunoglobulin heavy constant mu, transthyretin, fibrinogen alpha chain, fibrinogen beta chain, and fibrinogen gamma chain.

Optionally, additional biomolecules may be separated from the mixture using one or more additional MOFs and forming one or more additional encapsulated biomolecule@MOF composites, which are then also separated from unencapsulated biomolecules. In an embodiment, the method further comprises, after the encapsulated biomolecule@MOF composites are separated, adding a) a second organic ligand and b) second metal ions or a second metal ion precursor able to form the second metal ions, to the aqueous solution, where the second organic ligand and second metal ions surround one or more additional biomolecules and form the one or more additional encapsulated biomolecule@MOF composites. The one or more additional encapsulated biomolecule@MOF composites are then separated from remaining biomolecules in the aqueous solution that are not encapsulated and optionally analyzed. The second organic ligand may be the same or different from the first organic ligand, and the second metal ions may be the same or different than the first metal ions. In an embodiment, the second organic ligand is different from the first organic ligand, and the second metal ions are the same or different than the first metal ions.

One aspect of the invention separates high-abundance or interfering proteins from a biological fluid to enhance the detection and characterization of lower-abundance or harder to detect proteins. This allows the lower-abundance or harder to detect proteins to be more easily detected and accurately analyzed, for example, by MS techniques. This is particularly useful in blood samples that contain highly abundant plasma proteins as well as lower-abundant proteins and and protein biomarkers.

An embodiment of the invention provides a method for analyzing proteins in a blood sample comprising the step of encapsulating one or more proteins in the blood sample within a metal-organic framework (MOF), thereby forming encapsulated protein@MOF composites, where encapsulating the one or more proteins comprises adding a) an organic ligand, and b) metal ions or a metal ion precursor able to form the metal ions, to the blood sample, where the organic ligand and metal ions surround each of the one or more proteins and form the encapsulated protein@MOF composites. The encapsulated protein@MOF composites are separated from proteins that are not encapsulated, thereby generating a blood sample fraction that does not contain the one or more encapsulated proteins. Optionally, one or more analytical techniques are performed on the blood sample fraction that does not contain the one or more encapsulated proteins. Preferably, the blood sample is a blood serum or blood plasma sample.

Preferably, mass spectrometry (MS) analysis is performed on the remaining proteins in the blood sample that are not encapsulated. In an embodiment, the method further comprises collecting the separated encapsulated protein@MOF composites, degrading the MOF, recovering the one or more proteins, and performing one or more analytical techniques on the recovered one or more proteins. Suitable analytical techniques include, but are not limited to, MS, liquid chromatography (LC), LC-MS, electrophoresis, X-ray crystallography, NMR spectroscopy, Western blotting, small angle x-ray scattering (SAXS), Fourier-transform infrared spectroscopy (FTIR), and quantitative analysis.

Preferably, the one or more proteins that are separated from the blood sample are high-abundance proteins or proteins that prevent accurate detection or analysis of low-abundance or hard to detect proteins in the sample. In an embodiment, the one or more proteins that are encapsulated and separated from the blood sample comprise one or more proteins selected from the group consisting of: albumin, complement C3, serotransferrin, alpha-2-macroglobulin, alpha-1-antitrypsin, apolipoprotein A-I, apolipoprotein A-II, haptoglobin, immunoglobulin heavy constant gamma 1, immunoglobulin heavy constant gamma 2, immunoglobulin heavy constant gamma 3, immunoglobulin heavy constant gamma 4, immunoglobulin heavy constant alpha 1, immunoglobulin heavy constant alpha 2, immunoglobulin heavy constant mu, transthyretin, fibrinogen alpha chain, fibrinogen beta chain, and fibrinogen gamma chain. In an embodiment, at least 40%, at least 50%, at least 60%, at least 70%, at least 80%, at least 90%, or at least 95% of the one or more proteins are removed from the blood sample.

Optionally, additional proteins may be separated from the blood sample using one or more additional MOFs and forming one or more additional encapsulated protein@MOF composites, which are then also separated from the blood sample. In an embodiment, the method further comprises, after the encapsulated protein@MOF composites are separated, adding a) a second organic ligand and b) second metal ions or a second metal ion precursor able to form the second metal ions, to the aqueous solution, where the second organic ligand and second metal ions surround one or more additional proteins and form the one or more additional encapsulated protein@MOF composites. The one or more additional encapsulated protein@MOF composites are then separated from remaining proteins in the blood sample that are not encapsulated and are optionally analyzed. The second organic ligand may be the same or different from the first organic ligand, and the second metal ions may be the same or different than the first metal ions. In an embodiment, the second organic ligand is different from the first organic ligand, and the second metal ions are the same or different than the first metal ions.

In an embodiment, the one or more proteins to be encapsulated are protein fragments generated by digesting proteins in the blood sample, such as by adding a chemical or enzymatic digesting agent to the blood sample prior to adding the organic ligand and metal ions or the metal ion precursor. In an embodiment, after separation, the encapsulated proteins and/or unencapsulated proteins are digested, such as by adding a chemical or enzymatic digesting agent. Preferably, the digestion step is performed before analysis of the encapsulated proteins and/or unencapsulated proteins.

In an embodiment, encapsulating the one or more biomolecules or proteins comprises mixing the organic ligand and metal ions with the one or biomolecules or proteins for 5 minutes to 24 hours, preferably 1 to 24 hours, 1 to 8 hours, 1 to 4 hours, 1 to 2 hours, 20 minutes to 1 hour, 15 minutes to 1 hour, or 5 minutes to 4 hours. Preferably, the organic ligand and metal ions are mixed with the one or more biomolecules or proteins at a temperature between 4° C. and 40° C., preferably between 10° C. and 40° C., between 15° C. and 30° C., or between 18° C. and 25° C.

The encapsulated biomolecules-MOF composites and protein@MOF composites may be separated from the aqueous solution and blood sample using any separation technique known in the art, including but not limited to centrifugation and filtration.

An embodiment of the invention provides a kit for separating biomolecules, such as proteins, in an aqueous solution comprising:
a first metal-organic framework (MOF) reagent comprising a first organic ligand and first metal ion precursor able to form first metal ions in the aqueous solution, where the first organic ligand and first metal ions are able to selectively form a first MOF around one or more biomolecules in the aqueous solution; and
a second MOF reagent comprising a second organic ligand and a second metal ion precursor able to form second metal ions in the aqueous solution, where the second organic ligand and second metal ions are able to selectively form a second MOF around one or more different biomolecules in the aqueous solution. Preferably, the second organic ligand is different from the first organic ligand, and the second metal ions are the same or different than the first metal ions.

In an embodiment, the first and second organic ligands are each present in an amount so as to generate a concentration between 0.01 M to 10.0 M when added to the aqueous solution, preferably between 0.05 M to 5.0 M, between 0.05 M to 3.0 M, between 0.1 M to 2.0 M, between 0.3 M to 1.8 M, or between 0.5 M to 1.5 M. In an embodiment, the first and second metal ion precursors are each present in an amount so as to generate a concentration between 0.01 M to 5 M when added to the aqueous solution, preferably between 0.01 M to 2 M, between 0.02 M to 1 M, between 0.02 M to 0.5 M, or between 0.3 M to 0.1 M. In an embodiment, the organic ligands and metal ion precursors are added to the aqueous solution to produce a final volume of 5 ml, preferably 2 ml, 1 ml, 0.5 ml or 0.1 ml.

Optionally, the first metal ion precursor and the first organic ligand are present in an amount so as to generate between a 1:1 and 1:30 molar ratio when added to the aqueous solution, preferably between a 1:1 and 1:10 molar ratio, a 1:2 and 1:8 molar ratio, a 1:1 and 1:2 molar ratio, or between a 1:2 and 1:5 molar ratio. Similarly, the second metal ion precursor and the second organic ligand are optionally present in an amount so as to generate between a 1:1 and 1:10 molar ratio when added to the aqueous solution, preferably between a 1:2 and 1:8 molar ratio, or between a 1:2 and 1:5 molar ratio.

The organic ligand used in any of the embodiments described herein, may be any organic ligand able to interact with metal ions and the surface of a biomolecule and form a MOF in solution (see, for example, Xie et al., Adv. Sci. 2020, 7(4): 1901758; Hieu et al., ChemSusChem 2024, e202401568; Akpinar et al., J. Am. Chem. Soc. 2024, 146 (8): 5108-5117; and Hsu et al., ACS Appl. Mater. Interfaces 2021, 13 (44): 52014-52022). In embodiments described herein, two or more organic ligands may be mixed together to form the MOF. For example, the MOF may comprise 90% of 2-methylimidazole and 10% imidazolate-2-carboxyaldehyde, where both organic ligands are able to coordinate with the metal ions.

In an embodiment, the organic ligand has the formula:

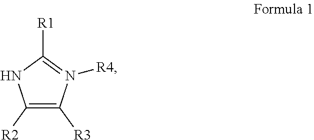

wherein R1, R2, and R3, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group, and
R4 is absent or is selected from the group consisting of a phosphate group, sulphate group, and C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group,
wherein, optionally, any two of R1-R4 together form a heterocyclic group, aryl groups, or heteroaryl group. Optionally, one or more C₁-C₁₂ alkyl groups are haloalkyl groups comprising one or more halogen atoms. It should be noted that if R4 is present, the double bond between the nitrogen atom and the neighboring carbon atom will be reduced to a single bond.

In an embodiment, R1, R2, and R3 in Formula 1, individually from each other, are selected from the group consisting of a hydrogen, phosphate group, sulphate group, nitro group, halogen, C₁-C₆ alkyl group, C₂-C₆ alkenyl group, C₁-C₆ alkoxy group, C₁-C₆ aldehyde group, C₂-C₆ ether group, C₂-C₆ ester group, and C₁-C₆ amino group. In an embodiment, R4 is absent or is selected from the group consisting of a phosphate group, sulphate group, nitro group, halogen, C₁-C₆ alkyl group, C₂-C₆ alkenyl group, C₁-C₆ alkoxy group, C₁-C₆ aldehyde group, C₂-C₆ ether group, C₂-C₆ ester group, and C₁-C₆ amino group.

In an embodiment, R1, R2, and R3 in Formula 1, individually from each other, are selected from the group consisting of a hydrogen, phosphate group, sulphate group, nitro group, halogen, C₁-C₄ alkyl group, C₂-C₄ alkenyl group, C₁-C₄ alkoxy group, C₁-C₄ aldehyde group, C₂-C₄ ether group, C₂-C₄ ester group, and C₁-C₄ amino group. In an embodiment, R4 is absent or is selected from the group consisting of a phosphate group, sulphate group, nitro group, halogen, C₁-C₄ alkyl group, C₂-C₄ alkenyl group, C₁-C₄ alkoxy group, C₁-C₄ aldehyde group, C₂-C₄ ether group, C₂-C₄ ester group, and C₁-C₄ amino group.

Preferably, R1, R2, and R3 in Formula 1, individually from each other, are selected from the group consisting of a hydrogen and a C₁-C₄ alkyl group, which is optionally a haloalkyl group comprising one or more halogen atoms. Preferably, R4 is absent or is selected from the group consisting of a C₁-C₄ alkyl group, which is optionally a haloalkyl group comprising one or more halogen atoms. In a further embodiment, R1, R2, and R3, individually from each other, are selected from the group consisting of a hydrogen and C₁-C₂ alkyl groups and R4 is absent. In an embodiment, the organic ligand is 2-methylimidazole (2-Melm).

In an embodiment, the organic ligand has the formula:

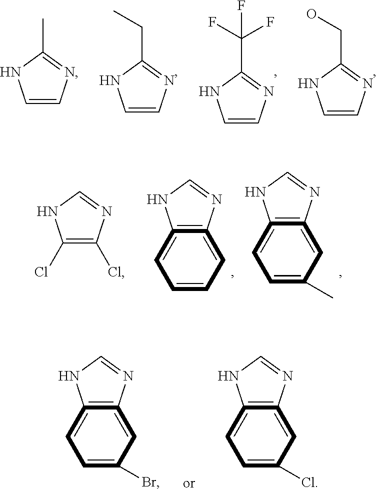

The R groups in imidazole-based organic ligands may be modified by adding a peptide or small molecule in order to increase or decrease surface interactions with proteins and other biomolecules, and thereby increase or decrease encapsulation. Accordingly, in an embodiment, the organic ligand has the formula:

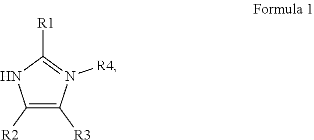

wherein R1, R2, R3 and R4, individually from each other, are selected from the group consisting of affinity peptides, charged peptides, positively or negatively charged hydrophilic molecules, hydrophobic molecules, and alkyl chains having terminally charged functional groups. It should be noted that if R4 is present, the double bond between the nitrogen atom and the neighboring carbon atom will be reduced to a single bond.

In an embodiment, the organic ligand has the formula:

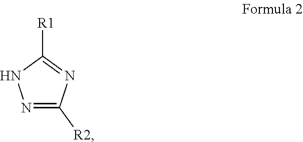

wherein R1 and R2, individually from each other, are selected from the group consisting of a hydrogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group.

In an embodiment, R1 and R2 in Formula 2, individually from each other, are selected from the group consisting of a hydrogen, C₁-C₆ alkyl group, C₂-C₆ alkenyl group, C₁-C₆ alkoxy group, C₁-C₆ aldehyde group, C₂-C₆ ether group, C₂-C₆ ester group, and C₁-C₆ amino group. Preferably, R1 and R2, individually from each other, are selected from the group consisting of a hydrogen and C₁-C₄ alkyl groups, wherein one or more of the C₁-C₄ alkyl groups are optionally haloalkyl groups comprising one or more halogen atoms.

In an embodiment, the organic ligand has the formula:

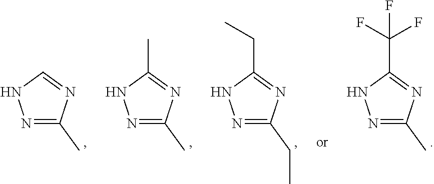

In an embodiment, the organic ligand has the formula:

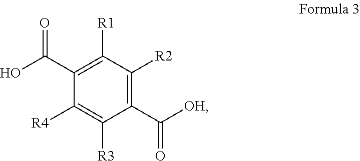

wherein R1, R2, R3 and R4, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group. In a further embodiment, R1, R2, R3 and R4, individually from each other, are selected from the group consisting of a hydrogen and C₁-C₁₂ alkyl groups, where one or more of the C₁-C₁₂ alkyl groups are haloalkyl groups comprising one or more halogen atoms.

In an embodiment, R1, R2, R3 and R4 in Formula 3, individually from each other, are selected from the group consisting of a hydrogen, phosphate group, sulphate group, nitro group, halogen, C₁-C₆ alkyl group, C₂-C₆ alkenyl group, C₁-C₆ alkoxy group, C₁-C₆ aldehyde group, C₂-C₆ ether group, C₂-C₆ ester group, and C₁-C₆ amino group. In a further embodiment, R1, R2, R3 and R4, individually from each other, are selected from the group consisting of a hydrogen and C₁-C₆ alkyl groups, where one or more of the C₁-C₆ alkyl groups are haloalkyl groups comprising one or more halogen atoms.

In an embodiment, R1, R2, R3 and R4 in Formula 3, individually from each other, are selected from the group consisting of a hydrogen, phosphate group, sulphate group, nitro group, halogen, C₁-C₄ alkyl group, C₂-C₄ alkenyl group, C₁-C₄ alkoxy group, C₁-C₄ aldehyde group, C₂-C₄ ether group, C₂-C₄ ester group, and C₁-C₄ amino group. In a further embodiment, R1, R2, R3 and R4, individually from each other, are selected from the group consisting of a hydrogen and C₁-C₄ alkyl groups, wherein one or more of the C₁-C₄ alkyl groups are optionally haloalkyl groups comprising one or more halogen atoms.

In an embodiment, the organic ligand has the formula:

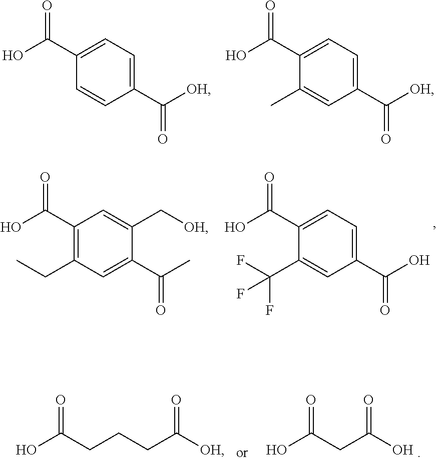

The metal ions used in any of the embodiments described herein, may be any metal ion able to interact with an organic ligand and the surface of a biomolecule and form a framework in solution.

In an embodiment, the metal ions are selected from the group consisting of zinc ions, iron ions, cobalt ions, copper ions, zirconium ions, aluminum ions, nickel ions, calcium ions, chromium ions, vanadium ions, titanium ions, magnesium ions, and combinations thereof. Preferably, the metal ions are selected from the group consisting of zinc ions, iron ions, cobalt ions, copper ions, zirconium ions, aluminum ions, nickel ions, calcium ions, and combinations thereof.

Examples of MOFs useful in the present invention include, but are not limited to, Zeolitic Imidazolate Frameworks, such as ZIF-8 (which comprises Zn and 2-methylimidazole), ZIF-67 (which comprises Co and 2-methylimidazole), ZIF-90 (which comprises Zn and imidazolate-2-carboxyaldehyde), as well as UiO-66, UiO-67, UiO-68 (which comprise Zr⁴⁺ clusters and amino acid modulators), and HKUST-1 (which comprises Cu²⁺ and imidazolate-2-carboxyaldehyde).

### BRIEF DESCRIPTION OF THE DRAWINGS

FIG. 1 illustrates protein-directed growth of protein@ZIF-8 biocomposites for selective protein encapsulation and removal of abundant proteins to enable integrated plasma proteomics analysis. (A) Scheme of selective biocomposite nucleation and encapsulation of abundant proteins in ZIF-8 results in two protein fractions: uncaptured (UC) fraction with remaining low-abundance proteins in the supernatant and captured (C) abundant acidic proteins in the precipitate. (B) After biocomposite growth, SDS-PAGE is utilized to quickly evaluate depletion performance. In parallel, the protein fractions are processed for analysis by top-down and bottom-up mass spectrometry (MS). (C) Schematic showing charge-mediated Zn²⁺ aggregation and nucleation of ZIF-8 on a representative abundant, acidic protein, and (D) lack of protein-mediated nucleation on a representative low-abundant, basic protein. These examples also illustrate that positively charged Zn²⁺ ions are attracted to negatively charged amino acid residues and repelled by positively charged residues.

FIG. 2 illustrates selective encapsulation of proteins during the in situ growth of protein@ZIF-8 biocomposites in a model standard protein mixture. (A) The standard protein mixture includes bovine serum albumin (BSA, pI 5.6), carbonic anhydrase (CA, pI 6.4), and cytochrome C (CYT C, pI 9.6) equally loaded to a total concentration of 0.75 mg/mL. After the ZIF-8 precursors are added to the protein mixture and the protein@ZIF-8 is grown for 2 h, centrifugation separates the uncaptured proteins (UC) and captured protein (C), which is dissolved using EDTA. (B) SDS-PAGE analysis of triplicate ZIF-8 growth in 0.75 mg/mL 3-protein mixture (LM). Uncaptured (UC), water wash (W) and captured (C) fractions were buffer exchanged with 50 mM HEPES 3 times. Each gel lane was loaded with 3 μg of protein and stained with Coomassie blue. (C) Protein fraction of BSA, CA, CYT C remaining in UC after standard protein (SP)@ZIF-8 growth at varying initial protein concentrations. Source gels are provided in the SI. (D) Powder X-ray diffraction (PXRD) patterns of SP@ZIF-8 grown in varying total protein concentrations, in comparison with the simulated pattern from dia crystal structure (CSD code: OFERUN01). Patterns are normalized to the most intense peak. (E) Scanning electron microscopy (SEM) image of the ZIF-8 grown in water and (F) SP@ZIF-8 grown in 0.75 mg/mL standard protein mixture in 50 mM HEPES buffer with morphology consistent with diamondoid (dia) crystal structure.

FIG. 3 illustrates in situ growth and selective depletion of abundant proteins via protein@ZIF-8 in human serum. (A) The HALT protease inhibitor was incubated in 2-Melm solution for 20 min before human serum and zinc precursor were added to initiate the growth of protein @ZIF-8 (20 min), then the sample was centrifuged and processed into UC and C fractions for further proteomics analysis. (B) Representative PXRD pattern of protein@ZIF-8 grown in 2 mg/mL human serum showing good agreement with SP@ZIF-8, ZIF-8 grown in water, and simulated ZIF-8 pattern (dia) (CSD code: OFERUN01). (C) SEM image of protein@ZIF-8 grown in 2 mg/mL human serum. (D) Fourier-transform infrared (FT-IR) spectra of 2 mg/mL serum@ZIF-8, 0.75 mg/mL SP@ZIF-8, ZIF-8 grown in water, and BSA. (E) Thermogravimetric analysis (TGA) of serum@ZIF-8. Mass fraction was normalized to the mass at 110° C. to account for water loss. (F) SDS-PAGE of triplicate protein@ZIF-8 growth in 2 mg/mL of human serum. Serum (S), UC, and C fractions were all buffer exchanged with 0.2% FA (6×). Each lane was loaded with 2 g of protein and stained with SYPRO Ruby.

FIG. 4 shows bottom-up proteomic analysis of serum depletion through ZIF-8 encapsulation. (A) Protein identification numbers among serum (S), uncaptured fraction (UC), and captured fraction (C) (n=3, error bars represent standard deviation). (B) Coefficient of variation and (C) Pearson correlations of protein intensities indicate high reproducibility of our approach. (D) Venn diagram comparing the proteins identified among the three sample groups. (E) Volcano plots showing fold changes in protein abundance due to ZIF-8 encapsulation (significance criteria: adjusted p-value ≤0.05 and |Log 2 Fold Change|≥1). Biomarkers associated with human diseases and high-abundance serum proteins are highlighted in red and labeled with gene names. (F) Unsupervised heatmap demonstrating z-score normalized intensities of proteins with significant abundance changes due to ZIF-8 encapsulation. The heatmap is divided into two clusters: Cluster 1 (grey) contains proteins with increased intensities in the uncaptured fraction, and Cluster 2 (black) contains proteins with decreased intensities in the uncaptured fraction. (G) Violin plots show the distribution of isoelectric points for proteins enriched (Cluster 1) and depleted (Cluster 2) in the uncaptured samples. (H) Depletion efficiency of the high-abundance serum proteins. The red line indicates 90% depletion efficiency.

FIG. 5 shows top-down proteomic analysis of serum depletion through ZIF-8 encapsulation. (A) Representative extracted ion chromatograms (EICs) of HSA, APOA1, and APOA2. (B) Quantification of intact protein abundance in serum (S), uncaptured fraction (UC), and captured fraction (C) (n=3, error bars represent standard deviations). (C) Representative deconvoluted MS spectra of HSA, APOA1, and APOA2. Roman numerals correspond to different proteoforms. HSA: (i) HSA-DA; (ii) HSA-L; (iii) HSA; (iv) HSA+Cys; (v) HSA+Glyc; (vi) HSA+Glyc+Cys. APOA1: (i) APOA1+2Ox; (ii) APOA1+3Ox. APOA2 dimer: (i) APOA2 [24-99][24-99]; (ii) APOA2 [24-100][24-99]; (iii) APOA2[24-100][24-100]. (D) Proteoform distribution of HSA, APOA1, and APOA2 in serum (blue bars) and captured fraction (green bars) (n=3, error bars represent standard deviations).

FIG. 6 shows detection and characterization of intact proteins enriched from human serum after depletion via ZIF-8 encapsulation. (A) Representative total ion chromatogram (TIC) and extracted ion chromatograms (EICs) of proteins in uncaptured fraction of human serum. (B) Deconvoluted mass spectra with most abundant masses corresponding to each labeled EIC. PDB: 1GYK (serum amyloid P component, APCS). (C) Comparison of EICs in serum (S), uncaptured fraction (UC), and captured fraction.

FIG. 7 shows estimated % BSA encapsulation in ZIF-8. The BSA@ZIF-8 composites were grown at in 1.5 mL water with 1 mg of BSA and a 4:1 ratio of 2-Melm:Zn at different total precursor concentrations. (Con. 1: 1.33 M 2-Melm and 0.33 M Zn, Con. 2: 0.67 M 2-Melm and 0.17 M Zn, Con. 3: 0.33 M 2-Melm and 0.083 M Zn). 50 μL aliquots were removed from growth supernatant after centrifugation at each time point, quenched with 50 μL of 100 mM EDTA and diluted for Bradford protein assay. High encapsulation (>60%) strongly coincides with MOF formation. As 2-Melm can interfere with protein assay readings, this is a rough estimate of the protein remaining in solution.

FIG. 8 shows SDS-PAGE of protein@ZIF-8 grown with and without buffer. ZIF-8 was grown using Con 2 (see FIG. 7) in a 5-protein mixture system including BSA (pI, 5.6), ovalbumin (OA, pI, 5.2, 42.7 kDa), CA (pI 6.4, 30.0 kDa), CYT C (pI 9.5, 11.7 kDa), and lysozyme (LZ, pI 9.4, 16.2 kDa). Proteins were equally loaded at 0.25 mg/mL for a total protein concentration of 1.25 mg/mL. Each lane of the 4-15% gradient gel was equally loaded by equal volume of supernatant before (LM) and after growth (8 μL) and stained with Coomassie blue. BSA and OA were entirely encapsulated in all conditions eventually, but the growth proceeded faster in HEPES and TRIS buffer. MOF was grown in 0.66 M 2-Melm and 0.167 M zinc acetate dihydrate.

FIG. 9 shows SDS-PAGE of uncaptured (UC) fraction after ZIF-8 growth in 3-protein solutions with varying starting total protein concentrations. Protein remaining in solution (uncaptured, UC) after 2 h of in situ ZIF-8 growth in 50 mM HEPES (pH 7.4), the amounts (ng) of BSA, CA, and CYT C were calculated and normalized to protein fraction within each lane. Total [protein] was varied while maintaining equal 1:1:1 mass loading for each protein. Protein concentration of UC fraction was determined using a Bio-Rad protein assay. 3 μg of proteins was loaded in each lane. Protein fraction of BSA, CA, and CYT C were calculated through ImageJ analysis using a standard curve of 4-15% gradient SDS-PAGE gel (top) and a 12% SDS-PAGE gel (bottom) stained with Coomassie blue.

FIG. 10 shows SDS-PAGE of uncaptured (UC) fraction after ZIF-8 growth in 3-protein solutions with varying BSA content. Protein remaining in solution (UC) after 2 h of in situ ZIF-8 growth in 50 mM HEPES (pH 7.4). The BSA % in the starting 3-protein solution was varied while maintaining 1 mg/mL total protein concentration. CA and CYT C were equally loaded to account for the remaining protein. Protein concentration of UC was determined using a Bio-Rad protein assay and run on a 4-15% gradient SDS-PAGE gels stained with Coomassie blue (A) 5 g of protein was loaded in each lane. (B) Difference in % composition of each protein in the UC versus the LM. (C) Percent change in protein fraction from LM to UC was calculated using ImageJ analysis using the LMs to construct a standard curve.

FIG. 11 illustrates theoretical framework structures of ZIF-8. (A) diamondoid (dia) (CSD code: OFERUN01) and (B) sodalite (sod) (CSD code: VELVOY).

FIG. 12 shows powder X-ray diffraction (PXRD) of SP@ZIF-8 biocomposite after EtOH wash. SP@ZIF-8 biocomposites grown in varying total protein concentrations after being washed with EtOH, in comparison with the simulated sodalite crystal structure to show the agreement. Patterns are normalized to the most intense peak.

FIG. 13 shows Fourier-transform infrared (FTIR) spectra of SP@ZIF-8 biocomposites, ZIF-8 and BSA. FTIR was performed on KBr pellets prepared with bovine serum albumin (BSA), ZIF-8 grown in water, and SP@ZIF-8 grown in varying total protein concentration composed of equally loaded BSA, carbonic anhydrase (CA), and cytochrome C (CYT C). The amide peak (1620-1730 cm¹) can be observed growing in at higher starting total protein concentration.

FIG. 14 shows overlaid total ion chromatograms (TICs) of top-down serum proteomics analysis. Protein fractions, including human serum (S), uncaptured (UC), and captured (C), were analyzed with three technical replicates. TIC traces between replicates were nearly identical.

### DETAILED DESCRIPTION OF THE INVENTION

### Definitions

As used herein, the term “biomolecules” refers to an organic substance produced by cells and living organisms, including but not limited to are carbohydrates, lipids, nucleic acids, proteins, glycoproteins, amino acids, glycolipids, fatty acids, saccharides, metabolites, vitamins, hormones, and combinations thereof.

The term “encapsulated” and “captured” refers to a biomolecule that is at least partially contained or bound within a metal-organic framework (MOF).

The term “metal ion precursor” refers to a salt or other chemical compound that contains a metal atom and generates a metal ion when dissolved in an aqueous solution. Common metal ion precursors include metal nitrates, chlorides, acetates, and metal oxides (for example, zinc acetate dihydrate).

The terms “protein” and “polypeptide” are used synonymously in the present description and refer to a class of compounds composed of amino acid residues chemically bonded together by amide bonds (or peptide bonds). Proteins and polypeptides are polymeric compounds comprising at least two amino acid residues or modified amino acid residues. Proteins can be modified, such as post-translational modifications or co-translational modifications. Modifications can be naturally occurring or non-naturally occurring, including modifications generated by chemical synthesis. Modifications to amino acids in proteins include, but are not limited to, phosphorylation, glycosylation, lipidation, prenylation, sulfonation, hydroxylation, acetylation, methylation, methionine oxidation, alkylation, acylation, carbamylation, iodination and the addition of cofactors. The proteins and polypeptides used herein further include compositions generated by degradation of larger proteins, for example by enzymatic digestion. Proteins include, for example, polypeptides comprising 10 to 5,000 amino acid units, optionally for some embodiments 10 to 3,000 amino acid units, 20 to 2,000 amino acid units, 50 to 1,000 amino acid units, and 50 to 800 amino acid units.

The term “alkyl” refers to a monoradical of a branched or unbranched (straight-chain or linear) saturated hydrocarbon and to cycloalkyl groups having one or more rings. Alkyl groups as used herein include those having from 1 to 12 carbon atoms, preferably having from 1 to 6 or 1-4 carbon atoms. Alkyl groups include small alkyl groups having 1 to 2 carbon atoms. Alkyl groups include medium length alkyl groups having from 4-10 carbon atoms. Alkyl groups include long alkyl groups having more than 10 carbon atoms, particularly those having 10-20 carbon atoms. Cycoalkyl groups include those having one or more rings. Cyclic alkyl groups include those having a 3-, 4-, 5-, 6-, 7-, 8-, 9-, 10-, 11- or 12-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group linked to oxygen and can be represented by the formula R—O. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups.

The term “alkenyl” refers to a monoradical of a branched or unbranched unsaturated hydrocarbon group having one or more double bonds and to cycloalkenyl groups having one or more rings wherein at least one ring contains a double bond. Alkenyl groups include those having 1, 2 or more double bonds and those in which two or more of the double bonds are conjugated double bonds. Alkenyl groups include those having from 2 to 20 carbon atoms, preferably having from 2 to 12 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those having one or more rings. Cyclic alkenyl groups include those in which a double bond is in the ring or in an alkenyl group attached to a ring. Cyclic alkenyl groups include those having a 3-, 4-, 5-, 6-, 7-, 8-, 9-, 10-, 11- or 12-member carbon ring and particularly those having a 3-, 4-, 5-, 6- or 7-member ring. The carbon rings in cyclic alkenyl groups can also carry alkyl groups. Cyclic alkenyl groups can include bicyclic and tricyclic alkyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogens replaced with one or more fluorine atoms.

Optional substitution of any alkyl and alkenyl groups includes substitution with one or more of the following substituents: halogens, —CN, —COOR, —OR, —COR, —OCOOR, —CON(R)₂, —OCON(R)₂, —N(R)₂, —NO₂, —SR, —SO₂R, —SO₂N(R)₂ or —SOR groups. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted.

Optional substituents for alkyl and alkenyl groups include among others:
—COOR where R is a hydrogen or an alkyl group or an aryl group and more specifically where R is methyl, ethyl, propyl, butyl, or phenyl groups all of which are optionally substituted;
—COR where R is a hydrogen, or an alkyl group or an aryl groups and more specifically where R is methyl, ethyl, propyl, butyl, or phenyl groups all of which groups are optionally substituted;
—CON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;
—OCON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;
—N(R)₂ where each R, independently of each other R, is an alkyl group, acyl group or an aryl group and more specifically where R is methyl, ethyl, propyl, butyl, or phenyl or acetyl groups all of which are optionally substituted; or R and R can form a ring which may contain one or more double bonds.
—SR, —SO₂R, or —SOR where R is an alkyl group or an aryl groups and more specifically where R is methyl, ethyl, propyl, butyl, phenyl groups all of which are optionally substituted; for —SR, R can be hydrogen;
—OCOOR where R is an alkyl group or an aryl groups;
—SO₂N(R)₂ where R is a hydrogen, an alkyl group, or an aryl group and R and R can form a ring;
—OR where R is H, alkyl, aryl, or acyl; for example, R can be an acyl yielding —OCOR* where R* is a hydrogen or an alkyl group or an aryl group and more specifically where R* is methyl, ethyl, propyl, butyl, or phenyl groups all of which groups are optionally substituted.

Specific substituted alkyl groups include haloalkyl groups, particularly trihalomethyl groups and specifically trifluoromethyl groups. As used herein, the term “halo” refers to a halogen group such as a fluoro (—F), chloro (—Cl), bromo (—Br) or iodo (—I).

As used herein, the term “mass spectrometry” (MS) refers to an analytical technique for the determination of the elemental composition of an analyte. Mass spectrometric techniques are useful for elucidating the chemical structures of analytes, such as proteins, peptides and other chemical compounds. The mass spectrometry principle consists of ionizing analytes to generate charged species or species fragments and measurement of their mass-to-charge ratios. Conducting a mass spectrometric analysis of an analyte results in the generation of mass spectrometry data relating to the mass-to-charge ratios of the analyte and analyte fragments. Mass spectrometry data corresponding to analyte ion and analyte ion fragments is presented in mass-to-charge (m/z) units representing the mass-to-charge ratios of the analyte ions and/or analyte ion fragments. In tandem mass spectrometry (MS/MS or MS2), multiple rounds of mass spectrometry analysis are performed to obtain structural information about molecules, such as peptides and proteins. For example, samples containing a mixture of proteins and peptides can be ionized and the resulting precursor ions separated according to their mass-to-charge ratio. Selected precursor ions can then be fragmented by MS/MS and further analyzed according to the mass-to-charge ratio of the fragments.

As used herein, the term “mass spectrometer” refers to a device that ionizes a sample, separates the resulting ions based on their mass-to-charge ratio (m/z), and detects their mass and relative abundance. Tandem mass spectrometry (MS/MS, or MS²) refers to the process of selecting a precursor ion, fragmenting it, and analyzing the resulting product ions. MS/MS can be performed mostly in time (within a single mass analyzer) or in space (using multiple physical mass analyzers).

### Overview

The detection and characterization of protein biomarkers and other biomolecules in biological fluids are critical for early disease detection and improved patient outcomes. Liquid chromatography-mass spectrometry (LC-MS) provides comprehensive protein characterization, but the detection of low-abundance protein biomarkers is often limited by the presence of highly abundant proteins, such as in plasma where human serum albumin (HSA) constitutes about 70-90% of the plasma proteome. As a result, depleting these abundant proteins is essential for effective protein analysis and biomarker discovery.

Current methods for plasma protein depletion include immunoaffinity columns, protein precipitation, and synthetic materials. Immunoaffinity-based depletion uses antibodies immobilized on columns or spin-columns to selectively bind and remove abundant proteins, making it widely used in blood proteomics. Protein precipitation exploits differences in solubility to isolate abundant proteins, followed by solid-phase extraction (SPE) to prepare samples for further analysis. Synthetic materials such as combinatorial peptide ligands and nanoparticle protein coronas have also been developed for plasma fractionation, enabling the identification of more proteins and proteoforms in serum. However, these methods face limitations such as high costs, increased sample handling requirements, reduced throughput, and inconsistent reproducibility. Therefore, there is an urgent need for more efficient and accessible techniques for depleting proteins and other biomolecules in biological fluids.

Metal-organic frameworks (MOFs) present a promising alternative for proteomics applications due to their simple synthesis, tunability, low cost, biocompatibility, and high surface area. MOFs interact with proteins through metal coordination, ligand affinity, and modifications, making them effective for peptide enrichment. Unlike other nanoparticles, MOFs can be dissolved for easy recovery, avoiding harsh elution conditions incompatible with mass spectrometry (MS). Recent advances, such as protein encapsulation within MOF pores or defects, have shown stability benefits and potential for separating proteins in complex mixtures. Zeolitic imidazolate framework-8 (ZIF-8) is particularly suited for in situ protein encapsulation, enabling selective protein separation based on encapsulation efficiency without requiring extensive pre-synthesis.

The present invention provides a novel method for selectively depleting high-abundance proteins from complex biological samples, such as human blood serum and plasma, to enhance the detection and characterization of low-abundance protein biomarkers. This method uses in situ synthesis of protein-encapsulating MOFs, particularly Zeolitic Imidazolate Framework-8 (ZIF-8), which forms selectively around high-abundance plasma proteins like HSA. By leveraging the specific surface chemistry interactions of these proteins with metal ions and organic linkers, the MOFs grow around the proteins, isolating them within their porous structures while leaving lower-abundance proteins in solution for proteomic analysis.

This selective encapsulation provides a streamlined, high-throughput, and gentle approach that reduces the need for costly, time-consuming depletion methods like immunoaffinity chromatography. The MOF-based method offers consistent depletion of abundant proteins without harsh elution steps, preserving the integrity of protein samples for both bottom-up and top-down mass spectrometry-based proteomics. The MOF material is easily dissolved under mild, MS-compatible conditions, allowing for easy recovery and analysis of encapsulated and unbound proteins, potentially in their native states.

As discussed further in the Examples below, the present invention addresses several key limitations in current methods for protein depletion useful for proteomics, offering significant improvements in efficiency, cost, reproducibility, and the depth of analysis.

Efficiency and Throughput: Current methods such as immunoaffinity depletion and protein precipitation often involve time-consuming, multi-step processes that increase sample handling requirements and limit throughput. The MOF-based approach provides a streamlined, high-throughput solution by selectively encapsulating abundant proteins directly within the framework during in situ synthesis. This reduces the need for multiple preparation steps and accelerates the depletion process.

Cost-Effectiveness: Traditional depletion methods, especially immunoaffinity-based strategies, can be costly due to the need for specialized reagents, antibodies, and materials. MOFs are low-cost, biocompatible, and easy to synthesize, making this method more affordable and accessible for broader use in clinical and research applications.

Improved Reproducibility: Current depletion strategies can lack consistency due to variability in antibody binding, solubility differences in protein precipitation, or the complex nature of synthetic material-based approaches. The simple and facile MOF-based depletion method offers greater reproducibility by utilizing well-defined interactions between proteins and the MOF structure, providing more consistent results across different samples.

Gentler Elution Conditions: Many current depletion methods require harsh conditions (e.g., surfactants, high temperatures, or strong chemicals) for elution, which can affect the integrity of proteins or peptides and make the process incompatible with mass spectrometry (MS). The MOF material used in this method grows and dissolves under mild, MS-compatible conditions, preserving sample integrity for more accurate downstream analysis, potentially allowing native proteomic analysis.

The methods and kits described herein can be applied in biomedical research, clinical labs, and the pharmaceutical industry. In clinical settings, the present invention could aid in early disease detection, accurate diagnosis, and personalized treatment strategies by identifying disease-related biomarkers for targeted therapies. In the biomedical research and pharmaceutical industry, the present invention could also be used to accelerate drug discovery by isolating novel biomarkers, validating drug targets, and improving proteomic analysis sensitivity, thus supporting the development of targeted therapies and advancing precision medicine.

### EXAMPLES

### Example 1—Protein Depletion Using In Situ Protein@-MOF Synthesis

Protein biomarkers in human serum provide critical insights into various physiological conditions and diseases, enabling early diagnosis, prognosis, and personalized treatment. However, detecting low-abundance protein biomarkers is challenging due to the presence of highly abundant proteins that make up ~99% of the plasma proteome. Here, the use of in situ metal-organic-framework (MOF) growth in serum is used to effectively deplete highly abundant serum proteins for integrated proteomic analysis. Through biomolecule-mediated nucleation of a zeolitic imidazolate framework (ZIF-8), abundant plasma proteins are selectively encapsulated within ZIF-8 and removed from serum via centrifugation, leaving a depleted protein fraction in the supernatant. Bottom-up proteomics analysis confirmed significant depletion of the topmost abundant proteins, many at depletion levels exceeding 95%. Such depletion enabled the identification of 277 total proteins in the supernatant (uncaptured) fraction in a single-shot analysis, including 54 uniquely identified proteins, 12 drug targets, and many potential disease biomarkers. Top-down proteomics characterization of the captured and uncaptured protein fractions at the proteoform-level confirmed this method is not biased toward any specific proteoform of individual proteins. These results demonstrate that in situ MOF growth can selectively and effectively deplete high-abundance proteins from serum in a simple, low cost, one-pot synthesis to enable integrated top-down and bottom-up proteomic analysis of serum protein biomarkers.

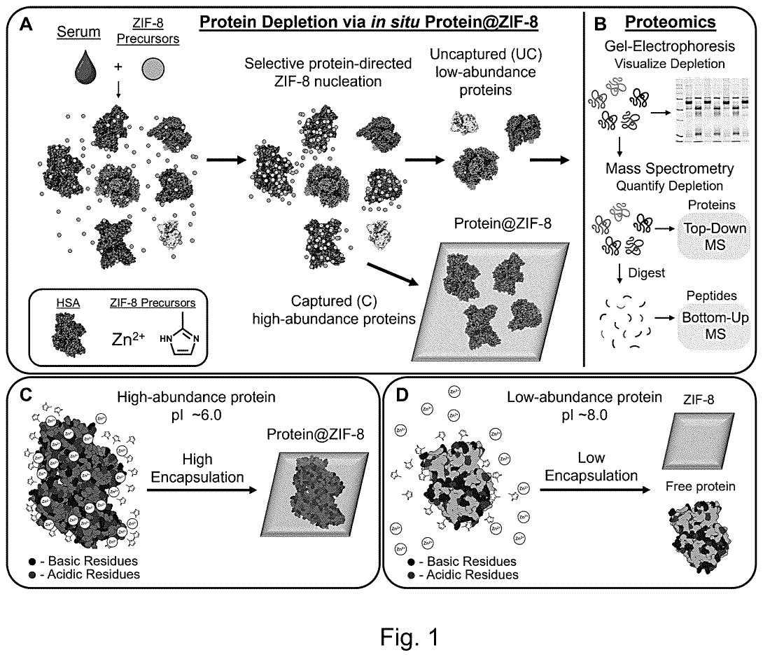

In this example, in situ ZIF-8 biocomposite growth was used and the selective protein encapsulation efficiency was leveraged to deplete highly abundant plasma proteins, including human serum albumin (HSA), from human serum (FIG. 1, panel A) for the first time to enable effective subsequent proteomic analysis (FIG. 1, panel B). By simply mixing the metal and organic precursors in a protein mixture, ZIF-8 selectively nucleates around abundant plasma proteins (FIG. 1, panel C), leaving behind lower-abundance proteins that do not sufficiently concentrate the Zn²⁺ and organic ligand precursor to nucleate growth (FIG. 1, panel D). Protein depletion through protein@ZIF-8 growth is applicable for both bottom-up and top-down proteomics analysis workflow, the two complementary MS-based proteomics approaches. Bottom-up proteomics focuses on analyzing peptides derived from protein digestion,⁶⁵⁻⁶⁷ and top-down proteomics can enable direct analysis of intact proteins for comprehensive proteoform characterization.^{5,68,69} The bottom-up proteomics analysis of serum (S) and uncaptured (UC) fraction showed 98% depletion of HSA and over 95% for the majority of the topmost abundant proteins. The removal of these high-abundance proteins enabled the identification of over 200 total proteins in the uncaptured fraction, including dozens of uniquely identified proteins, drug targets, and disease biomarkers. In addition, top-down MS characterization results suggest that the removal of these proteins is not biased towards any specific proteoforms belonging to the same protein.

### Selective Encapsulation and Depletion of Standard Proteins

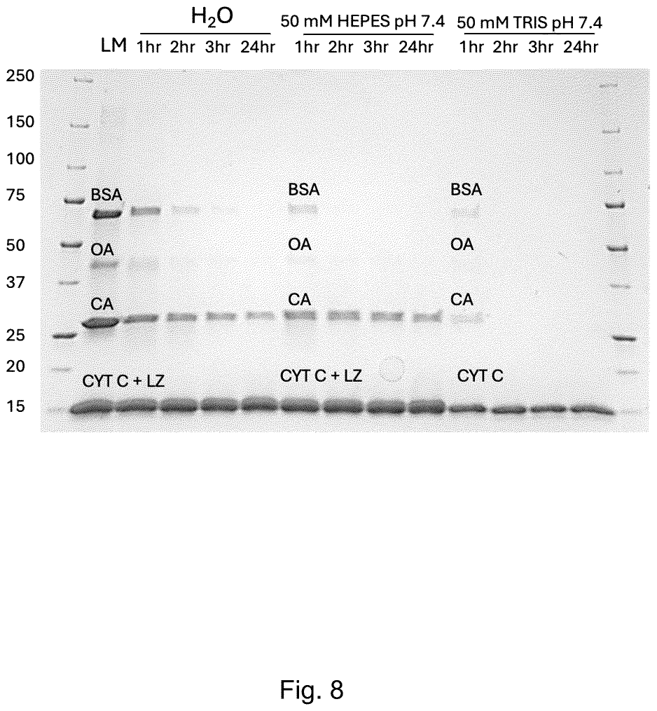

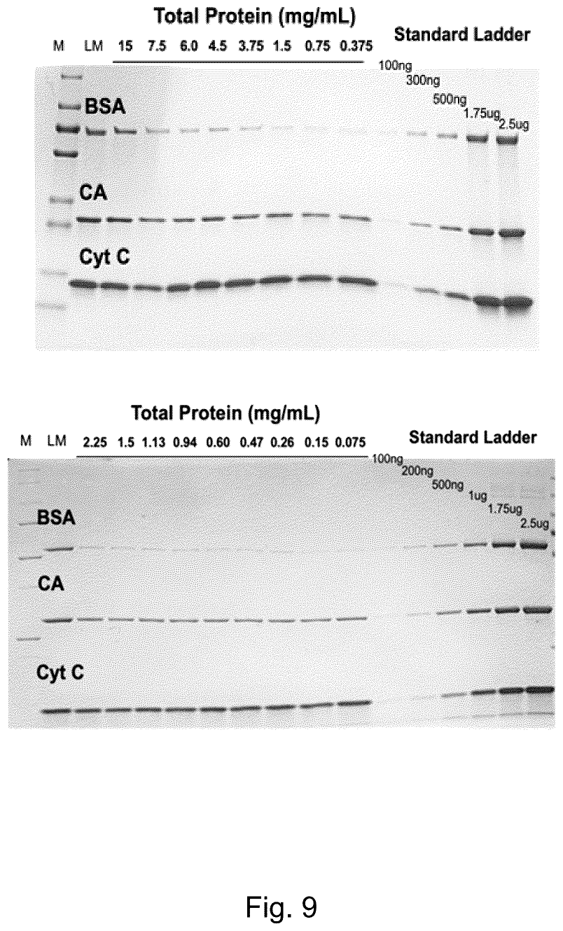

Protein encapsulation in ZIF-8 was first studied using the standard protein bovine serum albumin (BSA) alone. Zeolitic imidazolate framework-8 (ZIF-8) samples were initially prepared in water using zinc acetate dihydrate (Zn) and 2-methylimidazole (2-Melm) at a 1:4 molar ratio to minimize protein unfolding that can occur at higher ratios of 2-Melm.⁷⁰ The amount of BSA encapsulation in ZIF-8 grown at different precursor concentrations over time were measured using a BSA Bradford assay (FIG. 8). Significant (>50%) BSA encapsulation coincided with cloudy-white precipitation associated with ZIF-8 formation, indicating BSA depletion is not due to the presence of the Zn or 2-Melm precursor alone. The presence of BSA also resulted in slightly faster precipitation; however, precursor concentration had a more profound impact on growth rate. To minimize the total growth time, future growths were done at 1.33 M 2-methylimidazole (2-Melm) and 0.33 M zinc acetate dihydrate (Zn). Moreover, 50 mM of 4-(2-hydroxyethyl)-1-piperazineethanesulfonic acid (HEPES) buffer at pH 7.4 (FIG. 9) was added during the ZIF-8 growth to maintain physiological pH in the supernatant. Interestingly, the use of HEPES buffer facilitated protein depletion and biocomposite formation: the level of protein depletion achieved in water after 24 h could be achieved within only 2 h in HEPES buffer; therefore, all future growths were done in 50 mM HEPES buffer.

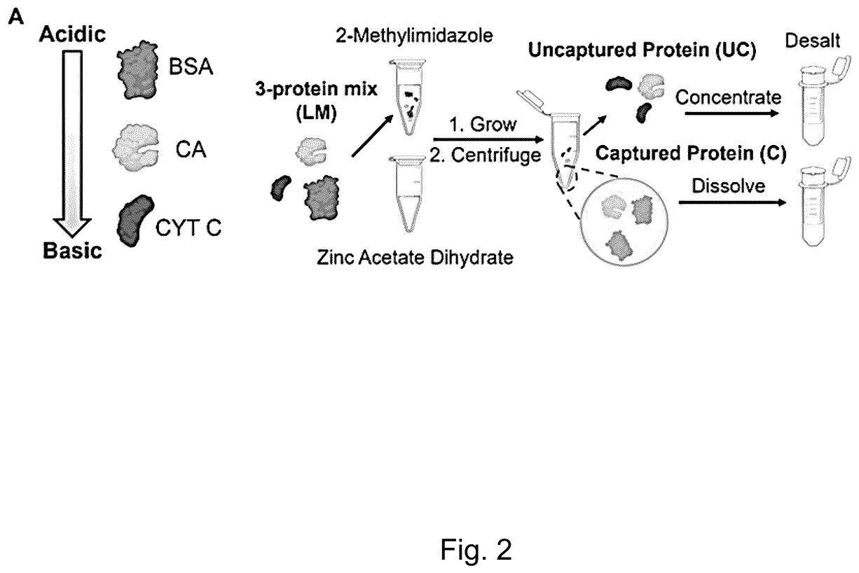

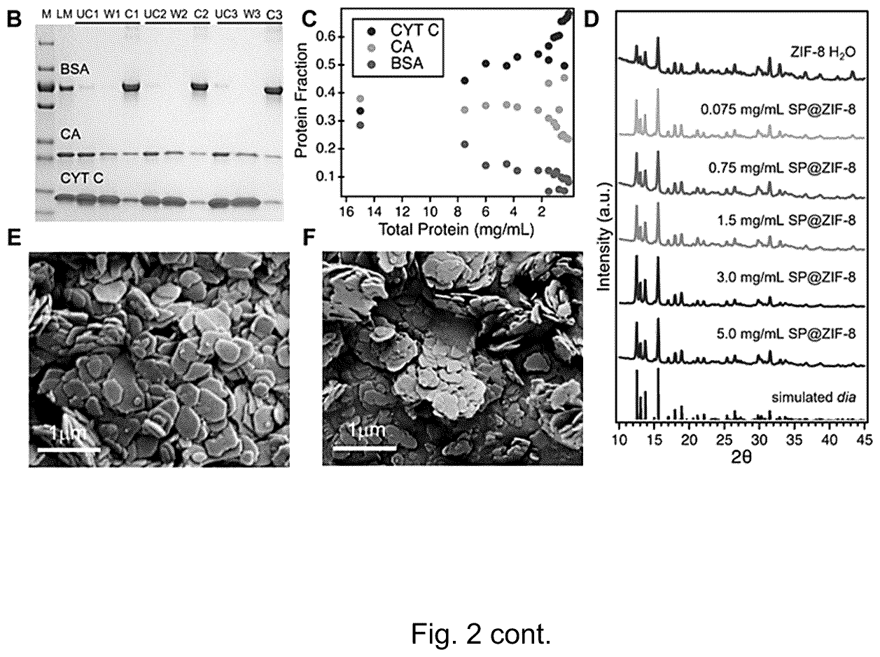

Next, the efficacy and reproducibility of protein depletion were evaluated in a standard protein (SP) mixture composed of BSA (pI 5.6, 66.5 kDa), carbonic anhydrase (CA, pI 6.4, 30.0 kDa), and cytochrome C (CYT C, pI 9.5, 12.3 kDa) equally loaded at 0.25 mg/mL in HEPES buffer (FIG. 2). For a standard depletion, 0.75 mg/mL total protein was briefly incubated in a 2-Melm solution containing 50 mM HEPES buffer. A separately prepared Zn solution was added, and standard protein@ZIF-8 biocomposite (SP@ZIF-8) was grown for 2 hours (FIG. 2, panel A). The biocomposites that formed were removed from the uncaptured proteins (UC) through centrifugation and then washed with water (W). The proteins captured in ZIF-8 (C) were released by dissolving the biocomposites in EDTA solution. The depletion was evaluated through SDS-PAGE analysis of each protein fraction (FIG. 2, panel B), which clearly showed that BSA was significantly depleted in the uncaptured fraction while CYT C was enriched relative to the loading mixture (LM) in each replicate. Previous works have shown protein encapsulation in ZIF-8 has a dependence on protein isoelectric point and more acidic proteins generally show increased encapsulation efficiency.^{59,63,71} The results from this model standard protein mixture support this trend, as encapsulation is selective toward BSA, the most acidic protein in the 3-protein mixture.

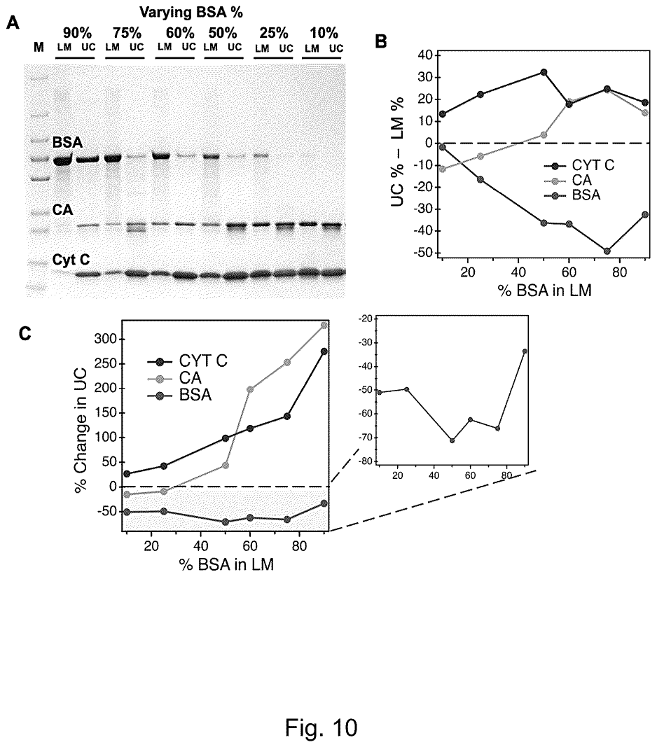

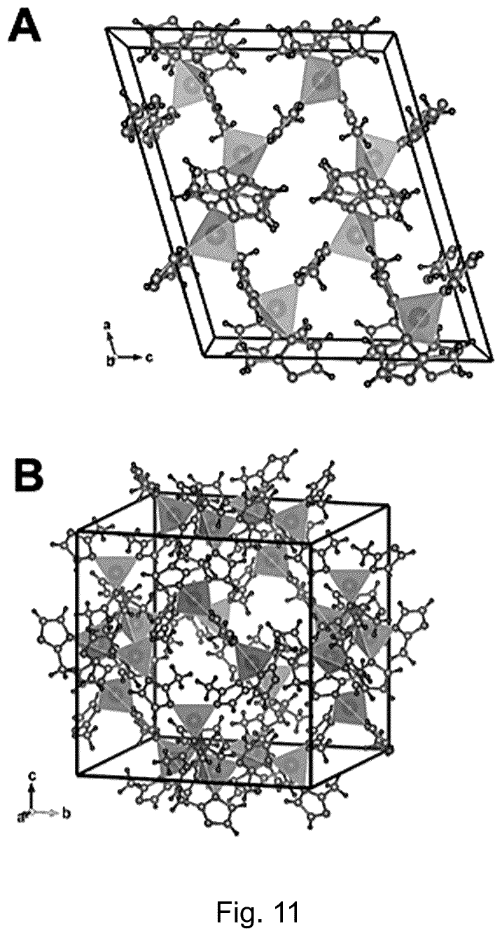

To further probe how protein concentration influences depletion efficiency, the protein fraction was examined as the total protein concentration decreased while maintaining MOF precursor concentration (FIG. 2, panel C, and FIG. 10). BSA encapsulation and depletion relative to CYT C and CA increases as the total protein concentration decreases and levels out at 2 mg/mL. Further, at concentrations below 2 mg/mL, the less acidic CA starts to get depleted. To simulate the concentration of albumin in human serum (HSA), the percentage of BSA were varied in the protein mixture from 10-90% (FIG. 11) and observed that the depletion efficiency of BSA was highest when BSA was between 50-75% of total protein, indicating depletion efficiency is higher when proteins are abundant. Further, the enrichment efficiency of CYT C and CA increases significantly at higher BSA percentages, while CA becomes depleted in growth with <50% BSA. These initial experiments revealed differential encapsulation efficiency of abundant acidic proteins and suggested the potential for in situ ZIF-8 growth to deplete abundant proteins in serum which are largely acidic.

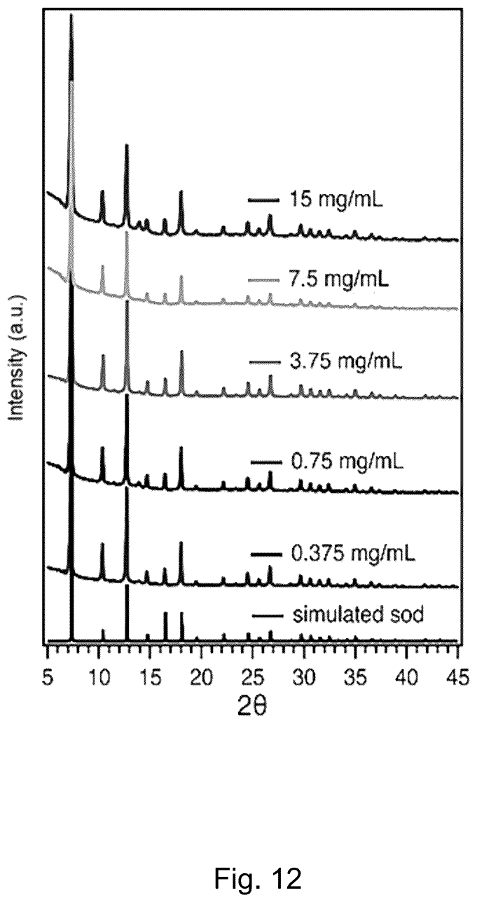

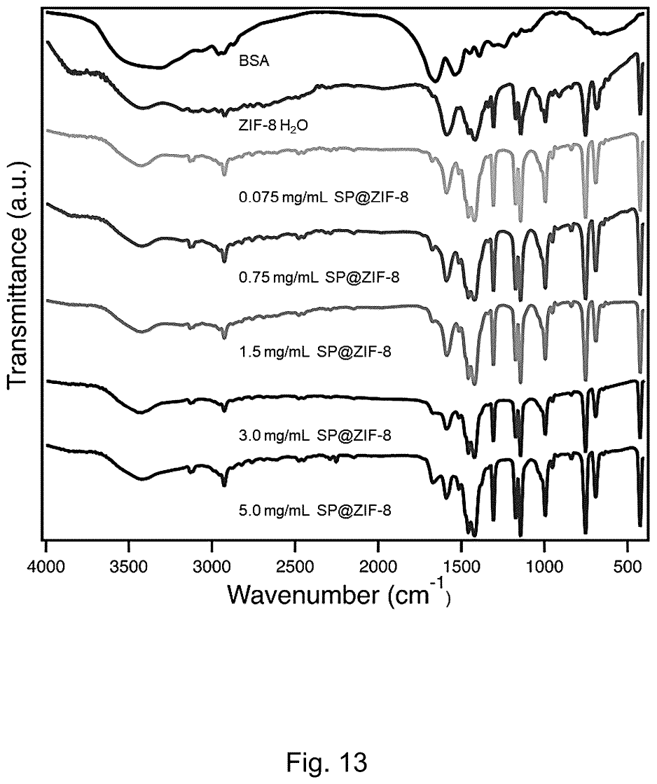

The ZIF-8 biocomposites formed in the standard protein mixture were characterized using powder X-ray diffraction (PXRD) and scanning electron microscopy (SEM). PXRD patterns (FIG. 2, panel D) showed ZIF-8 grown in water and SP@ZIF-8 biocomposites grown in 50 mM HEPES buffer exhibited diamondoid (dia) structure when they were washed with water (2×) and allowed to air-dry overnight. SEM images revealed irregular plate-like structures for both growths (FIG. 2, panels E and F), consistent with the dia crystal structure (FIG. 12, panel A). It should be noted that the crystal structure of these protein@ZIF-8 biocomposites is sensitive to the washing and aging procedures. If the MOFs were with ethanol (2×), the porous sodalite (sod) crystal structure was formed regardless of drying conditions, confirmed through PXRD (FIG. 12, panel B, FIG. 13).⁶¹

### Encapsulation and Depletion of High-Abundance Plasma Proteins

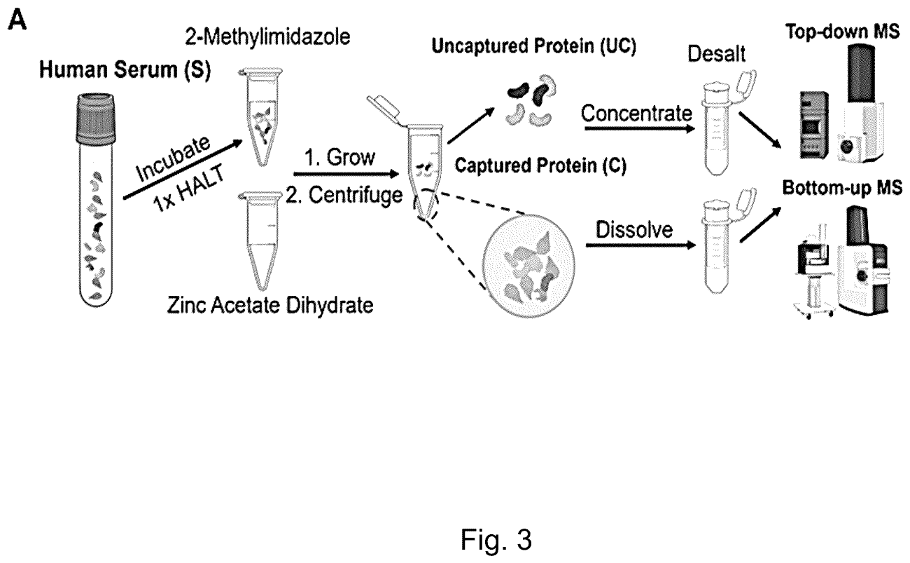

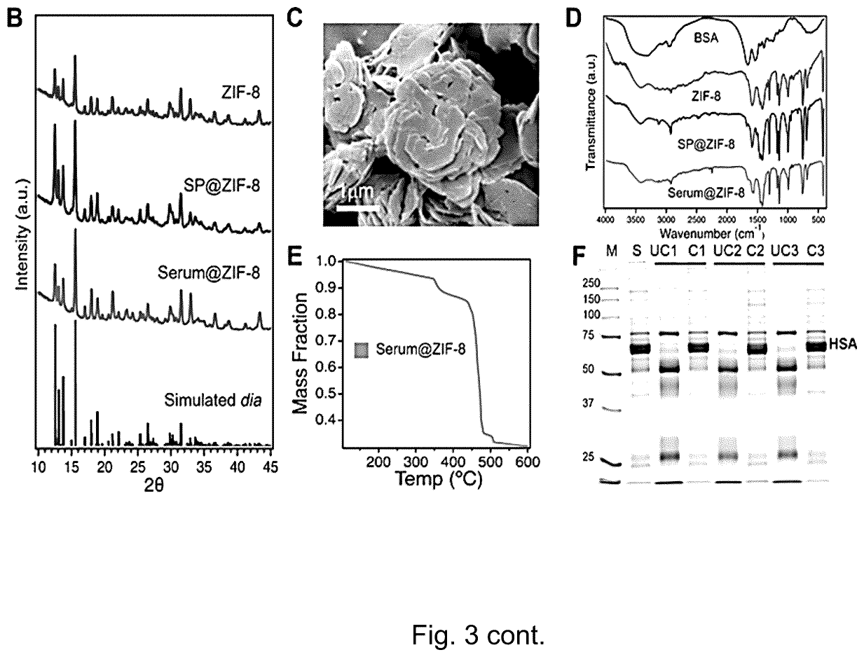

Next, ZIF-8 growth was conducted in human serum using a similar workflow as described above. The EDTA free HALT protease inhibitor cocktail was added during the growth to inhibit serum proteases and prevent proteolytic cleavage of proteins (FIG. 3, panel A). Protein@ZIF-8 biocomposite formation occurred more readily in human serum, likely due to the increased sample complexity, thus the growth time was shortened to 20 min to minimize protein oxidation.

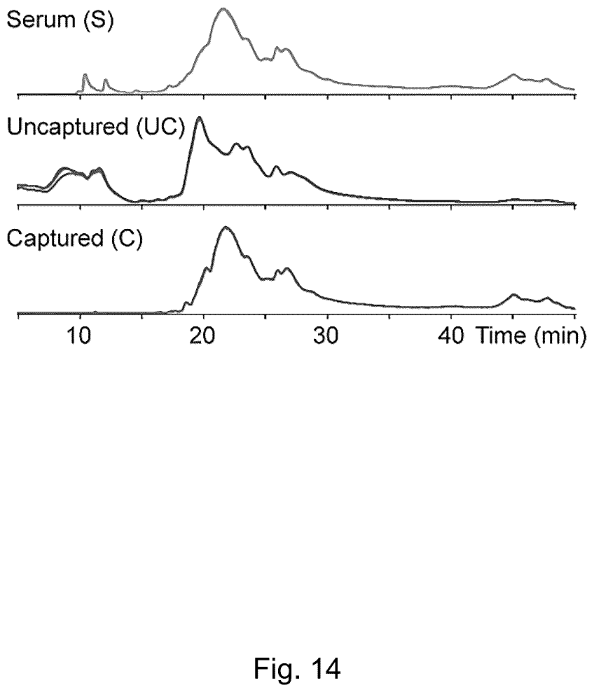

The protein@ZIF-8 biocomposites formed from serum also exhibited the diamondoid (dia) crystal structure by PXRD, just as the ZIF-8 grown in water and in the standard protein system (FIG. 3, panel B). SEM revealed the familiar plate-like morphology consistent with the dia crystal structure (FIG. 3, panel C); but with a more irregular shape and larger size than the ZIF-8 grown in water and SP@ZIF-8. Fourier-transform infrared spectroscopy (FT-IR) was performed on serum@ZIF-8, SP@ZIF-8 and ZIF-8 to probe protein encapsulation (FIG. 3, panel D, FIG. 14). All ZIF-8 samples exhibited the characteristic peak at 423-425 cm⁻¹ associated with Zn—N stretching of ZIF-8.⁷²⁻⁷⁴ Further, the broad amide peak from 1535-1720 cm⁻¹ associated with proteins increased in the serum@ZIF-8 biocomposites, confirming protein encapsulation within the ZIF-8. There was also significant broadening of the 3200-3700 cm⁻¹ region, which corresponds to N—H and O—H stretching indicating the increased presence of proteins and/or water in serum@ZIF-8.

Thermogravimetric analysis (TGA) of the serum@ZIF-8 biocomposites exhibited a 53.5% mass loss from 435-510° C., and a total mass loss of ~70% (FIG. 3, panel E). Additionally, there was a mass loss of ~10% from 340-430° C., which could correspond to the loss of the encapsulated proteins. These results are consistent with protein loading in previous reports of protein@ZIF-8 biocomposites as well as ZIF-8 with dia morphology.^{51,75,76}

Importantly, the protein depletion from serum by the in situ ZIF-8 growth and encapsulation was evaluated through SDS-PAGE analysis in triplicate (FIG. 3, panel F). It is very clear that the most abundant human serum albumin (HSA) is significantly depleted in the uncaptured fraction, as well as some other abundant protein bands. Furthermore, in the uncaptured (UC) fraction, some protein bands were visibly enriched with new proteins emerging, which were not visible in the original serum (S) sample. Additionally, proteins that were encapsulated by ZIF-8 appeared to be entirely recovered after ZIF-8 dissolution. This depletion is highly reproducible between replicates and the whole process can be completed in less than an hour.

### Bottom-Up Proteomics Analysis

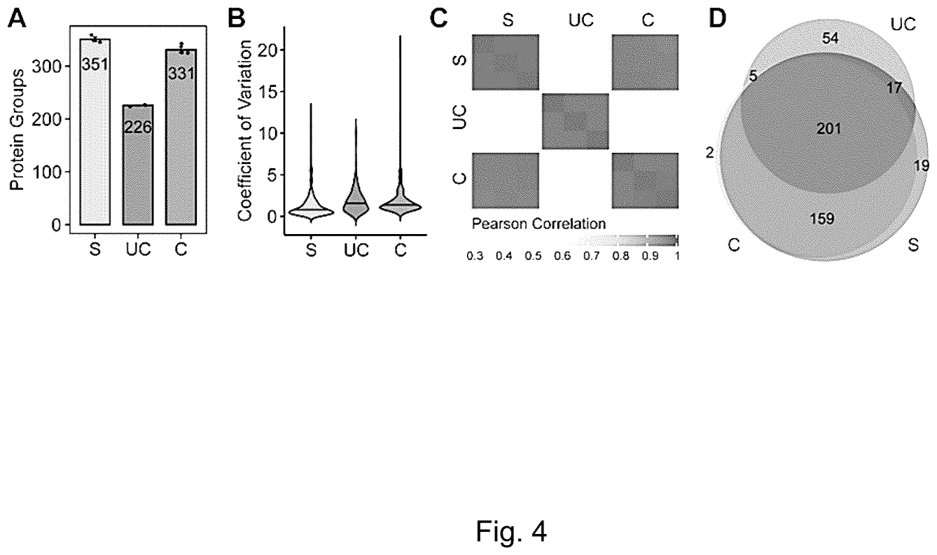

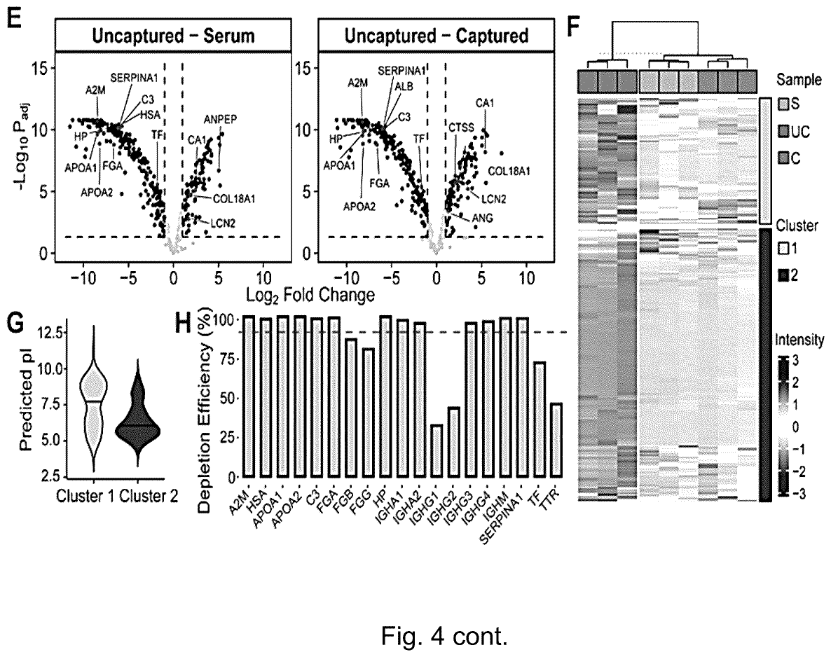

A bottom-up proteomic analysis was performed to characterize the different protein fractions and evaluate the performance of serum protein depletion via ZIF-8 encapsulation in triplicate samples. The photo-cleavable-surfactant, Azo,⁷⁷ was added to all samples to accelerate trypsin digestion prior to LC-MS/MS analysis.⁷⁸ The analysis identified 396 total proteins (351 proteins on average) in serum, 277 total proteins (226 proteins on average) in uncaptured, and 367 total proteins (331 proteins on average) in captured fractions (FIG. 4). The reproducibility of ZIF-8 encapsulation and LC-MS/MS analysis is demonstrated by the highly consistent total ion chromatograms and comparable protein identification numbers across replicates (FIG. 4, panel A). Reliable protein quantification is further supported by the uniformity of protein intensity distributions and low coefficients of variation (FIG. 4, panel B). Pearson correlation and principal component analysis reveal strong clustering of replicates within each group and clear separation between groups, highlighting the effects of ZIF-8 encapsulation on serum proteome composition (FIG. 4, panel C). A Venn diagram shows significant overlap in protein identifications between the captured fraction and serum, with a lower overlap for the uncaptured fraction (FIG. 4, panel D). Notably, 54 proteins were uniquely identified in the uncaptured fraction (Table 1).

TABLE 1

| | | |
|---|---|---|
| O15335 | CHAD | Chondroadherin |
| O60844 | ZG16 | Zymogen granule membrane protein 16 |
| O75368 | SH3BGRL | Adapter SH3BGRL |
| P00390 | GSR | Glutathione reductase, mitochondrial |
| P00441 | SOD1 | Superoxide dismutase [Cu—Zn] |
| P00533 | EGFR | Epidermal growth factor receptor |
| P01854 | IGHE | Immunoglobulin heavy constant epsilon |
| P02792 | FTL | Ferritin light chain |
| P04040 | CAT | Catalase |
| P04179 | SOD2 | Superoxide dismutase [Mn], mitochondrial |
| P05154 | SERPINA5 | Plasma serine protease inhibitor |
| P05164 | MPO | Myeloperoxidase |
| P05451 | REG1A | Lithostathine-1-alpha |
| P06744 | GPI | Glucose-6-phosphate isomerase |
| P07998 | RNASE1 | Ribonuclease pancreatic |
| P09668 | CTSH | Pro-cathepsin H |
| P10153 | RNASE2 | Non-secretory ribonuclease |
| P16930 | FAH | Fumarylacetoacetase |
| P23284 | PPIB | Peptidyl-prolyl cis-trans isomerase B |
| P61916 | NPC2 | NPC intracellular cholesterol transporter 2 |
| P68104 | EEF1A1 | Elongation factor 1-alpha 1 |
| Q10588 | BST1 | ADP-ribosyl cyclase/cyclic ADP-ribose hydrolase 2 |
| Q13228 | SELENBP1 | Methanethiol oxidase |
| Q14314 | FGL2 | Fibroleukin |
| Q16270 | IGFBP7 | Insulin-like growth factor-binding protein 7 |
| Q96AP7 | ESAM | Endothelial cell-selective adhesion molecule |
| A0A0A0MSI4 | IGHV1-45 | Immunoglobulin heavy variable 1-45 |
| Q9HD89 | RETN | Resistin |
| P12318 | FCGR2A | Low affinity immunoglobulin gamma Fc region receptor II-a |
| P08217 | CELA2A | Chymotrypsin-like elastase family member 2A |
| P84243 | H3-3A | Histone H3.3 |
| A0A075B614 | IGLV10-54 | Immunoglobulin lambda variable 10-54 |
| P02794 | FTH1 | Ferritin heavy chain |
| P68371 | TUBB4B | Tubulin beta-4B chain |
| P08238 | HSP90AB1 | Heat shock protein HSP 90-beta |
| P10809 | HSPD1 | 60 kDa heat shock protein, mitochondrial |
| A0A0C4DH39 | IGHV1-58 | Immunoglobulin heavy variable 1-58 |
| O43280 | TREH | Trehalase |
| P05186 | ALPL | Alkaline phosphatase, tissue-nonspecific isozyme |
| P63241 | EIF5A | Eukaryotic translation initiation factor 5A-1 |
| Q8WVN6 | SECTM1 | Secreted and transmembrane protein 1 |
| Q9P121 | NTM | Neurotrimin |
| P06732 | CKM | Creatine kinase M-type |
| P11279 | LAMP1 | Lysosome-associated membrane glycoprotein 1 |
| P62942 | FKBP1A | Peptidyl-prolyl cis-trans isomerase FKBP1A |
| O95267 | RASGRP1 | RAS guanyl-releasing protein 1 |
| P31151 | S100A7 | Protein S100-A7 |
| Q8NCC3 | PLA2G15 | Lysosomal phospholipase A and acyltransferase |
| P07951 | TPM2 | Tropomyosin beta chain |
| P17174 | GOT1 | Aspartate aminotransferase, cytoplasmic |
| O75594 | PGLYRP1 | Peptidoglycan recognition protein 1 |
| P12273 | PIP | Prolactin-inducible protein |
| Q86YZ3 | HRNR | Hornerin |
| P31944 | CASP14 | Caspase-14 |
| O15335 | CHAD | Chondroadherin |

Among these identified proteins, 12 are FDA-approved drug targets, such as EGFR, a key target in cancer therapy;⁷⁹ and FKBP1A, a target for preventing transplant rejection.⁸⁰ Several other proteins are also important disease biomarkers, including MPO and IGFBP7 which are associated with cardiovascular disease and could be promising targets for treatment of heart failure.⁸¹⁻⁸⁵

Using a limma-based differential expression analysis, significant changes in protein abundances resulting from ZIF-8 encapsulation were identified (FIG. 4, panel E). Notably, several high-abundance serum proteins exhibited substantially lower intensities in the uncaptured fraction. More importantly, increased intensities were observed for several key biomarkers, such as ANPEP (CD13), an ectoenzyme linked to tumor invasion and angiogenesis;⁸⁶ LCN2, an iron-trafficking protein involved in apoptosis, innate immunity, and renal development;⁸⁷ COL18A1, a precursor of endostatin that inhibits endothelial cell proliferation and angiogenesis;⁸⁸ CA1, a zinc-bound enzyme that serves as a target in glaucoma treatment;⁸⁹ CTSS, a thiol protease whose alteration is implicated in lymphoma and rheumatoid arthritis;⁹⁰ and ANG, a protein associated with prevalent hypertension.⁹¹ Using these differentially expressed proteins, unsupervised hierarchical clustering was performed, which revealed that the uncaptured fraction formed a distinct cluster with a significantly different protein abundance pattern compared to the captured and serum sample groups (FIG. 4, panel F). Two major protein clusters were identified: cluster 1, enriched in the uncaptured fraction due to the removal of high-abundance serum proteins, and cluster 2, composed of proteins captured by ZIF-8. Further analysis of the isoelectric points (pI) of these clusters showed that the median pI of proteins in cluster 1 was 7.9, in comparison with the 6.0 in cluster 2 (FIG. 4, panel G). This observation aligns with the established trend that ZIF-8 tends to capture acidic proteins, indicating that MOF encapsulation follows similar protein separation principles even in complex biological samples such as human serum.

To assess the efficacy of ZIF-8 encapsulation for serum protein depletion, bar charts of the 22 topmost high-abundance serum proteins were made. (FIG. 4, panel H). For example, the most abundant protein HSA showed a 51-fold decrease in intensity in uncaptured fraction post-ZIF-8 encapsulation, corresponding to a 98% depletion (Table 2).

TABLE 2

| | | | | Protein | Depletion |
|---|---|---|---|---|---|
| UniProt | | | Predicted | Intensity in | Efficiency |
| Accession | Gene | Protein Name | pI | Serum | (%) |
| P02768 | ALB | Albumin | 5.92 | 3.3E+09 | 98.1 |
| P01024 | C3 | Complement C3 | 6.02 | 6.2E+08 | 98.4 |
| P02787 | TF | Serotransferrin | 6.81 | 4.8E+08 | 71.1 |
| P01023 | A2M | Alpha-2-macroglobulin | 6.03 | 4.7E+08 | 99.7 |
| P01009 | SERPINA1 | Alpha-1-antitrypsin | 5.37 | 4.2E+08 | 98.6 |
| P02647 | APOA1 | Apolipoprotein A-I | 5.56 | 3.7E+08 | 99.7 |
| P00738 | HP | Haptoglobin | 6.13 | 3.0E+08 | 99.7 |
| P01857 | IGHG1 | Immunoglobulin heavy | 6.55 | 1.7E+08 | 31.8 |
| | | constant gamma 1 | | | |
| P01859 | IGHG2 | Immunoglobulin heavy | 6.11 | 9.8E+07 | 42.6 |
| | | constant gamma 2 | | | |
| P01876 | IGHA1 | Immunoglobulin heavy | 5.43 | 9.2E+07 | 97.3 |
| | | constant alpha 1 | | | |
| P02652 | APOA2 | Apolipoprotein A-II | 6.27 | 8.4E+07 | 99.6 |
| P01871 | IGHM | Immunoglobulin heavy | 5.8 | 7.5E+07 | 98.7 |
| | | constant mu | | | |
| P02766 | TTR | Transthyretin | 5.49 | 5.4E+07 | 45.1 |
| P02763 | ORM1 | Alpha-1-acid | 5.02 | 2.8E+07 | — |
| | | glycoprotein 1 | | | |
| P01861 | IGHG4 | Immunoglobulin heavy | 5.88 | 2.7E+07 | 96.6 |
| | | constant gamma 4 | | | |
| P01860 | IGHG3 | Immunoglobulin heavy | 6.5 | 2.5E+07 | 95.7 |
| | | constant gamma 3 | | | |
| P19652 | ORM2 | Alpha-1-acid | 5.03 | 7.7E+06 | — |
| | | glycoprotein 2 | | | |
| P01877 | IGHA2 | Immunoglobulin heavy | 5.42 | 1.1E+06 | 95.5 |
| | | constant alpha 2 | | | |
| P02671 | FGA | Fibrinogen alpha | 5.7 | 1.1E+06 | 98.9 |
| | | chain | | | |
| P02788 | LTF | Lactotransferrin | 8.5 | 2.7E+05 | — |
| P02675 | FGB | Fibrinogen beta chain | 8.54 | 1.3E+05 | 85.5 |
| P02679 | FGG | Fibrinogen gamma | 5.37 | 9.5E+04 | 79.4 |
| | | chain | | | |

In total, 13 out of these 22 proteins demonstrated >95% of depletion efficiency, followed by 6 proteins with depletion levels between 30-85%. The only proteins that showed slightly increased intensity in the uncaptured fraction are lactotransferrin, which has a high isoelectric point (8.5), and both alpha-1-acid and alpha-2-acid glycoproteins (pI 5.0). Based on their predicted isoelectric point (5.0) and experimentally determined isoelectric point (2.8-3.8),⁹² alpha-1-acid and alpha-2-acid glycoproteins are expected to have the highest encapsulation efficiency of the top 22 most abundant proteins. However, other structural factors likely influence encapsulation efficiency including the number of non-acidic Zn-binding residues (cysteine and histidine),⁶⁴ spacing between precursor coordinating sites, functional group accessibility and rigidity, and co-encapsulation of proteins. In this case, the extremely acidic nature of these proteins could result from their extensive glycosylation (45% of MW).⁹²⁹³ Despite their contribution to a negative surface change, it is possible these bulky glycans lack the rigidity and density necessary to promote nucleation of ZIF-8 while further preventing encapsulation by shielding the more rigid residues present on the protein backbone. These results suggest selective protein encapsulation in ZIF-8 involves mechanisms beyond simple electrostatic interactions, such as those observed in anion-exchange chromatography.^{15,16}

Compared to other common depletion strategies, this strategy shows comparable depletion effectiveness, with significantly higher removal rates shown for some proteins. For example, the present method depletes HSA with a higher depletion efficiency compared to the precipitation-based method (98% vs. 95%).¹⁹ This approach removes APOA1 and APOA2 with significantly higher removal rates compared to conventional immunoaffinity-based depletion (>99% vs. 47%, and >99% vs. 31%, respectively).⁹ Although recent studies in blood proteomes have achieved higher protein identification numbers, the results depend on sample types, cohort sizes, sample preparation methods, LC-MS/MS instrumentation, and MS acquisition settings in each study.¹⁰ Only commercial serum was used in this current study, but this MOF encapsulation strategy is able to be applied to disease-relevant plasma/serum samples and additional separation strategies could be used to expand the proteome coverage.^{27,94,95} Overall, this global proteomic analysis provides new insights into how ZIF-8 encapsulation alters protein abundances in serum and demonstrates its use as a simple, low cost, and high throughput serum depletion technique.

### Top-Down Proteomics Analysis

Top-down proteomics analysis was used to evaluate the effectiveness of ZIF-8 encapsulation for serum depletion. Samples were buffer exchanged into 0.2% formic acid and analyzed under non-reducing conditions using a high-resolution Impact II quadrupole time-of-flight mass spectrometer (Bruker Daltonics) coupled with a NanoAcquity ultra-high pressure LC system (Waters). Chromatography and instrument response remained consistent and reproducible throughout the analysis (FIG. 14). Three high-abundance serum proteins were identified with multiple proteoforms—6 from HSA, 2 from APOA1, and 3 from APOA2 (Table 3).

TABLE 3

| Protein | UniProt Accession | Proteoform name |
|---|---|---|
| HSA | P02768 | HSA − DA |
| | | HSA − L |
| | | HSA |
| | | HSA + Cys |
| | | HSA + Glyc |
| | | HSA + Cys + Glyc |
| APOA1 | P02647 | APOA1 + 20x |
| | | APOA1 + 30x |
| APOA2 | P02652 | APOA2 [24-99] [24-99] |
| | | APOA2 [24-100] [24-99] |
| | | APOA2 [24-100] [24-100] |

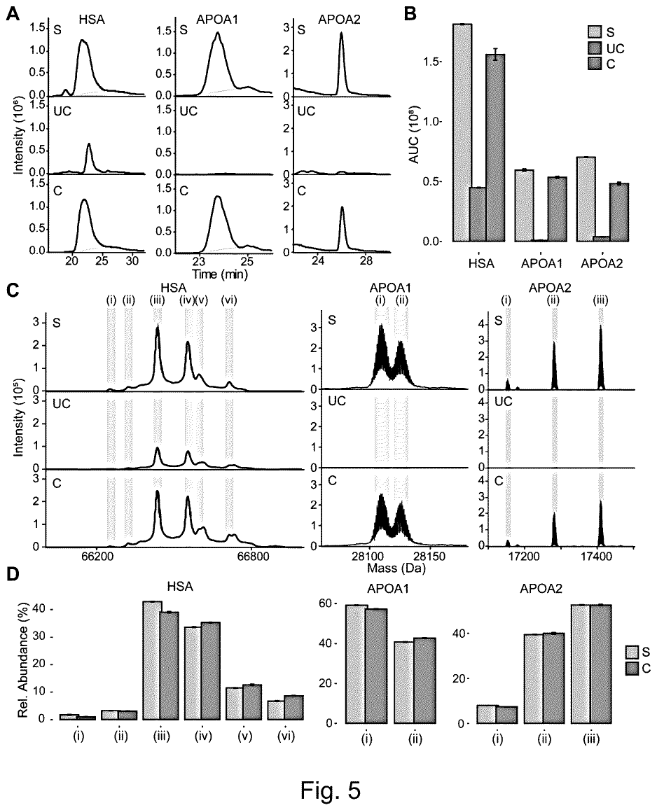

Extracted ion chromatograms (EICs) of the three proteins show similar peak intensities between serum and the captured fraction, whereas the intensities in the uncaptured fraction greatly decrease (FIG. 5, panel A). Quantification of protein abundance based on EICs showed that, after depletion, the signals of these three proteins decrease 4-fold, 58-fold, and 18-fold, respectively (FIG. 5, panel B). Importantly, deconvoluted top-down mass spectra illustrate that proteoforms derived from the same protein, including those with truncated variants, post-translational modifications (PTMs) such as oxidation, glycation, and cysteinylation, as well as protein complexes with disulfide bridges, show negligible influence on ZIF-8 encapsulation efficiency (FIG. 5, panel C). Moreover, quantification of the relative abundances of these proteoforms reveals that the proteoform distribution remains unchanged before and after ZIF-8 encapsulation. Online LC-MS/MS was used to characterize the APOA1 and APOA2 proteoforms.

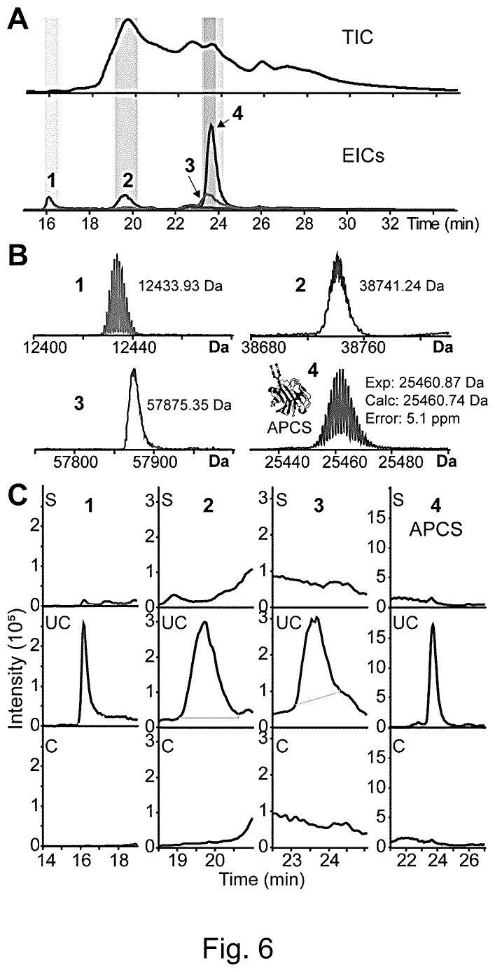

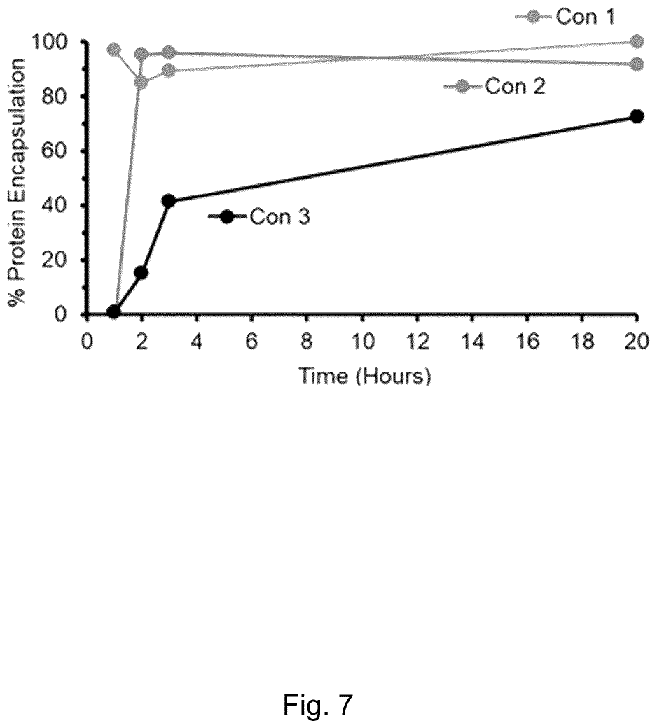

Aside from proteins depleted from serum, 4 proteins were enriched after removal of high-abundance proteins, including 12.4 kDa, 38.7 kDa, 57.9 kDa, and 25.5 kDa species (FIG. 6). Notably, the 25.5 kDa species was identified to be glycosylated serum amyloid P-component (APCS), a biomarker in Alzheimer's disease, liver fibrosis, and cardiovascular disease.⁹⁶ Online auto MS/MS was used to generate fragment ions that confirmed the protein identity. These results showed that selective protein encapsulation in ZIF-8 enables effective non-biased removal of high-abundance serum proteins with various proteoforms and enhances the detection and characterization of low-abundance disease biomarkers. A recent study reported an integrative method combining engineered nanoparticles²⁰⁻²² and gel-based prefractionation that led to the identification of high numbers of proteoforms, albeit with relatively high cost of the engineered nanoparticles, reduced throughput from multi-step fractionation, and the use of denaturing, surfactant-based elutions.²²

In contrast, the present MOF-based depletion method is simple, inexpensive, rapid (less than one hour) and fully compatible with top-down MS and proteomics. Moreover, many studies have shown that protein conformation and catalytic activity are preserved in these proteins encapsulation within MOFs;^{42,51,54,55,58} so it is conceivable that the MOF depletion method is mild (non-denaturing) enough to maintain native protein conformation in the UC fraction. On the other hand, magnetic nanoparticles functionalized with small-molecule or peptide affinity ligands have been developed to enrich biomarker proteins and protein complexes from human tissues in a targeted fashion to enable the comprehensive top-down MS and native top-down MS characterization of the proteoforms of these biomarkers.⁹⁷⁻⁹⁹ Yet the enrichment of endogenous protein biomarkers directly from human serum using such functionalized magnetic nanoparticles has remained challenging. Further increase in the biomarker enrichment efficiency in serum is achievable by integrating the simple MOF depletion workflow with such targeted nanoparticle enrichment method.

### CONCLUSION

The detection and characterization of low-abundance protein biomarkers in human plasma is hindered by the presence of highly abundant plasma proteins, which must be depleted by simple, low-cost, yet effective techniques to expand proteome coverage. MOF materials are used herein to provide a new, simple, low-cost, and effective method for the depletion of highly abundant plasma proteins through a one-pot in situ synthesis of protein@ZIF-8 biocomposites. Through selective protein-mediated nucleation of ZIF-8 in human serum, 19 out of the top 22 abundant proteins including HSA, APOA1, and APOA2 are encapsulated and significantly depleted in less than an hour. After serum protein depletion using ZIF-8 encapsulation, 277 total proteins were identified in the uncaptured (UC) protein fraction through bottom-up proteomics in single-shot mass spectrometry analysis, including 54 uniquely identified proteins, 12 drug target proteins (such as EGFR and FKBP1A), and many low abundance potential disease biomarkers that have been significantly enriched, including ANPEP (CD13), LCN2, COL18A1, CA1, CTSS, and ANG.

Top-down proteomics analysis further demonstrated that this depletion method does not alter proteoform distribution or stoichiometry, thereby enabling the characterization and quantification of proteoforms of low-abundance human serum biomarkers. These results demonstrate the potential for in situ MOF growth to fractionate the proteome effectively at low cost to enable integrated top-down and bottom-up proteomic analysis. This example provides a new tool for plasma proteomics and paves the way for more accurate, accessible and high throughput proteomic analysis and biomarker discovery in research and clinical settings.

### Example 2—Experimental Methods and Materials

### Chemicals and Reagents

2-Methylimidazole (2-Melm) was purchased from Santa Cruz Biotechnology (Santa Cruz, CA, USA), and zinc acetate dihydrate (CAS: 5970-45-6) was purchased from Neta Scientific Inc. (Hainesport, NJ, USA). Human serum from male AB plasma (USA origin, sterile filtered) was purchased from Millipore Sigma (CAT #H4522, Burlington, MA, USA). 4-15% Mini-PROTEAN TGX Precast Protein Gels (CAT #4561086) and XT 12% Bis-Tris Protein Gels (CAT #3450118) were purchased from Bio-Rad Laboratories (Hercules, CA, USA). All other reagents were purchased from Millipore Sigma (Burlington, MA, USA) or Thermo Fisher Scientific (Waltham, MA, USA) unless noted otherwise.

### ZIF-8 Synthesis in Water

For a 1.5 mL growth, various concentrations of 2-Melm (2.00 mmol) were dissolved in water (1.00 mL). Then, the corresponding zinc acetate dihydrate solution (0.50 mmol in 500 μL water) was added to the 2-Melm solution, vortexed for 5 seconds, and placed onto a rotator. The ZIF-8 was allowed to grow for 2 h then centrifuged at 20,000×g, washed with water (1.5 mL, 2×), and allowed to air-dry for materials characterization.

### ZIF-8 Synthesis in BSA

For a 1.5 mL growth, various concentrations of 2-Melm (2.00, 1.00, and 0.50 mmol) were dissolved in water (1.00 mL), and 1 mg of bovine serum albumin (BSA) was added to the 2-Melm solution. The solution was vortexed and incubated for ~1 min. Then, the corresponding zinc acetate dihydrate solution (0.50, 0.25, and 0.125 mmol in 500 μL water) was added to the 2-Melm solution in a 1:4 ratio, vortexed for 5 seconds, and placed onto a rotator. The protein@ZIF-8 biocomposites were allowed to grow for 24 h with intermittent centrifugation at 20,000×g to allow for sampling of the supernatant. The concentration of BSA in the supernatant was measured using a Bradford protein assay.

### ZIF-8 Synthesis in 3-Protein System

For a 0.5 mL growth, 2-Melm (0.667 mmol) were dissolved in water (284 μL), then 500 mM HEPES buffer (50 μL), pH 7.4, was spiked into the 2-Melm solution and vortexed. Then, carbonic anhydrase (CA), BSA, and cytochrome C (CYT C) were added to the 2-Melm solution at varying concentrations. The solution was vortexed and incubated for ~1 min. Then, the zinc acetate dihydrate solution (0.167 mmol in 166 μL water) was added to the 2-Melm solution, immediately vortexed for 5 seconds, and placed onto a rotator. The protein@ZIF-8 biocomposites were allowed to grow for 2 h followed by centrifugation at 20,000×g.

### ZIF-8 Synthesis in Human Serum

For a typical 1.0 mL growth, 2-Melm (1.33 mmol) was dissolved in water (566 μL), then 500 mM HEPES (100 μL), pH 7.4, was spiked into the 2-Melm solution and vortexed. 100×HALT protease inhibitor (10 μL) was added to the 2-Melm solution and vortexed and allowed to incubate for ~20 min. The protein concentration of human serum was determined using a Bio-Rad protein assay, and 2 mg of human serum was incubated in 50 mM EDTA for ~1 minute. Next, the human serum was spiked into the 2-Melm solution, vortexed for 5 seconds and allowed to incubate for ~10 min. Then, zinc acetate dihydrate solution (0.333 mmol in 333 μL water) was added to the 2-Melm solution, vortexed for 5 seconds, and placed onto a rotator. The biocomposites were allowed to grow for at least 20 min followed by centrifugation at 20,000×g. The resulting protein@ZIF-8 biocomposites were washed with 1.0 mL of water (2×) to remove proteins that were loosely bound to the MOF surface and air-dried for materials characterization.

### Materials Characterization

Powder X-ray diffraction (PXRD) measurements were performed on a Bruker D8 Advance diffractometer with a Cu Kα (λ=1.54178 Å) radiation source with scattering angles (2θ) of 5-45°. Scanning electron microscopy was performed using the Zeiss SUPRA 55VP Scanning Electron Microscope for all samples except the serum@ZIF-8 growth in 2 mg/mL human serum (FIG. 4, panel C), which was imaged using the Zeiss LEO 1530-1 FESEM. All MOF or protein@MOF samples were sputtered with Au for 60 seconds. Thermogravimetric analysis (TGA) was performed using a TA Instruments Q5 thermal analysis system under an O2 atmosphere and at a constant heating rate of 30.00° C./min from 100-600° C. The samples were first heated to 100° C. and held at that temperature for 8 min to remove adsorbed water and precursors loosely bound to the MOF surface. Fourier transform infrared spectroscopy (FTIR) measurements were performed on a Bruker Equinox 55 FT-IR spectrometer in the spectral range of 4,000 to 400 cm-1 at 2 cm-1 resolution on various samples in a potassium bromide (KBr) pellet.

### Sample Preparation for Gel Electrophoresis

Depletion in 3-protein mixture. For a 0.5 mL growth, protein@ZIF-8 biocomposites were synthesized following the procedure described above, and following centrifugation at 4° C. (5 min, 20,000×g), the supernatant (uncaptured fraction, UC) was collected and quenched with 500 mM EDTA (100 μL). Proteins that were loosely bound to the MOF surface were removed by washing with 0.5 mL water (2×). To release the proteins captured (C) in the MOF, 500 mM EDTA (500 μL) was added to the biocomposites and the mixture was vortexed and sonicated for 30 seconds. Once this captured (C) solution became clear, both UC and C fractions were buffer exchanged 3× into water using Amicon 10 kDa 0.5 mL molecular weight cut-off (MWCO) filters. Then the protein concentration was measured using a Bio-Rad protein assay (Bio-Rad, Hercules, CA, USA, Cat #5000006) using bovine serum albumin to generate a standard curve. For each experiment, depletion was evaluated using SDS-PAGE on the original standard protein mixture (LM), uncaptured (UC) and captured (C) fractions, and each lane had 3 μg of protein loaded onto a 4-15% Mini-PROTEAN@TGX gradient gel (Bio-Rad, Cat #4561085). The gels were stained with Coomassie blue.

### Sample Preparation for Gel Electrophoresis and Mass Spectrometry

Depletion in human serum. For a 1.0 mL growth, protein@ZIF-8 biocomposites were synthesized following the procedure described above, and following centrifugation at 4° C. (5 min, 20×g), the supernatant (uncaptured fraction, UC) was collected, and the MOF growth was quenched with 500 mM EDTA (200 μL)+Formic Acid (FA, 10 μL). Proteins that were loosely bound to the MOF surface were removed by washing with 1.0 mL water (2×). To release the proteins captured (C) in the MOF, 500 mM EDTA (1000 μL)+FA (10 μL) was added to the biocomposites and the mixture was vortexed and sonicated. Once this captured (C) solution became clear, the original human serum (S), UC and C fractions were buffer exchanged 6× into 0.2% FA using Amicon 10 kDa 0.5 mL MWCO filters. Following buffer exchange, the protein concentration was measured with a Bio-Rad protein assay (Bio-Rad, Hercules, CA, USA, Cat #5000006), using bovine serum albumin to generate a standard curve. Then all samples were compatible with top-down MS and gel electrophoresis. For each experiment, depletion was evaluated using SDS-PAGE on the serum, uncaptured (UC) and captured (C) fractions, and each lane had 2 μg (SYPRO stain) or 10 μg (Coomassie stain) of protein loaded onto a homemade 10% polyacrylamide gel. The gel was stained with SYPRO Ruby stain or Coomassie blue.

### Sample Preparation for Bottom-Up Mass Spectrometry Analysis

Serum (S), Captured (C), and Uncaptured (UC) were normalized to 1 mg/mL protein in 0.1% Azo surfactant in 25 mM ammonium bicarbonate (Cat #919233).1,2 Disulfide bonds were reduced with 10 mM TCEP at 37° C. for 30 min and alkylated with 30 mM 2-chloroacetamide in the dark for 30 min. Then Trypsin Gold (Promega, Madison, WI, USA) was added in a protein:protease ratio (wt/wt) of 50:1 and incubated for 24 h at 37° C. The digestion was quenched by acidifying the samples to pH 2 with formic acid (FA) and placing them on ice. After digestion, Azo was degraded by irradiation at 305 nm using a UVN-57 Handheld UV Lamp (Analytik Jena, Jena, TH, DEU) for 5 min,^{77,78,100} then the samples were spun at 20,000× g at 4° C. for 15 min, and then desalted using 100 μL Pierce C18 tips (Thermo Fisher Scientific). Desalted peptides were dried in a vacuum centrifuge and reconstituted in 0.1% FA. The peptide concentration was determined by A205 readings using NanoDrop. Samples were centrifuged again at 20,000×g at 4° C. for 30 min prior to analysis.

### Bottom-Up Proteomics Analysis

The timsTOF Pro mass spectrometer (Bruker Daltonics) was coupled with a C18 capillary column (25 cm length, 75 μm inner diameter, 1.6 μm particle size, 120 Å pore size; IonOpticks). 200 ng peptides were injected and separated at a flow rate of 400 nL/min using a 120-min gradient with 2-85% mobile phase B (mobile phase A: 0.1% FA; mobile phase B: 0.1% FA in acetonitrile) as previously reported.¹⁰⁰⁻¹⁰² Data was acquired under PASEF mode.¹⁰³ MS raw files were processed with FragPipe (version 22.0) using a UniProt human FASTA database UP000005640 (accessed on Jul. 25, 2024).¹⁰⁴ Trypsin was set as the digestion rule with a maximum of 2 missed cleavages. Carbamidomethylation on cysteine was set as a fixed modification, while variable modifications include methionine oxidation and protein N-termini acetylation. The false discovery rate was controlled to be less than 1% for peptide-spectrum matches and protein groups. Label-free quantification was performed with match-between-runs function enabled. After FragPipe processing, data were further analyzed using “DAPAR” and “DEP” packages for R (version 4.2.1).^{105,106} Protein quantities were filtered, Log 2-transformed, normalized, and imputed. Limma tests were performed to evaluate statistical significance, and Limma p-values were adjusted via Benjamini-Hochberg method. Proteins with FDR-adjusted p-values ≤0.05 and Log 2-fold change ≥1 were considered to show significant difference.

### Top-Down Proteomics Analysis

A high-resolution Impact II quadrupole time-of-flight mass spectrometer (Bruker Daltonics) was coupled with a NanoAcquity ultra-high pressure LC system (Waters). 2 μg proteins were injected and separated by reverse-phase liquid chromatography (RPLC) on a home-packed PLRP column (PLRP-S, 1000 Å pore size, 10 μm particle size, 250 μm inner diameter, 15 cm length; Agilent Technologies) at 60° C., following procedures reported in our previous publications.^{101,107-109} The gradient was 60 min with a flow rate at 12 μL/min using 10-95% mobile phase B (mobile phase A: 0.2% FA; mobile phase B: 0.2% FA in acetonitrile). Intact mass spectra were acquired at a scan rate of 1 Hz from 300-3000 m/z. Auto MS/MS was enabled to perform collisionally activated dissociation (CAD) for protein backbone fragmentation. Extracted ion chromatograms and deconvoluted MS spectra were generated using DataAnalysis software (version 4.3, Bruker Daltonics). The SNAP algorithm was used to determine the most abundant masses of individual proteoforms and their intensities. Tandem mass spectra (MS/MS) were analyzed using MASH Native software.¹¹⁰ The spectra were deconvoluted using eTHRASH with a signal-to-noise ratio of 3 and cutoff fit score of 70%. All fragment assignments were manually validated.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### CITED REFERENCES

(1) Roehrl, M. H.; Roehrl, V. B.; Wang, J. Y. Proteome-Based Pathology: The next Frontier in Precision Medicine. Expert Rev. Precis. Med. Drug Dev. 2021, 6, 1-4.
(2) Doll, S.; Gnad, F.; Mann, M. The Case for Proteomics and Phospho-Proteomics in Personalized Cancer Medicine. PROTEOMICS—Clin. Appl. 2019, 13, 1800113.
(3) Issaq, H. J.; Xiao, Z.; Veenstra, T. D. Serum and Plasma Proteomics. Chem. Rev. 2007, 107, 3601-3620.
(4) Geyer, P. E.; Holdt, L. M.; Teupser, D.; Mann, M. Revisiting Biomarker Discovery by Plasma Proteomics. Mol. Syst. Biol. 2017, 13, 942.
(5) Melby, J. A.; Roberts, D. S.; Larson, E. J.; Brown, K. A.; Bayne, E. F.; Jin, S.; Ge, Y. Novel Strategies to Address the Challenges in Top-Down Proteomics. J. Am. Soc. Mass Spectrom. 2021, 32, 1278-1294.
(6) Zhu, P.; Bowden, P.; Zhang, D.; Marshall, J. G. Mass Spectrometry of Peptides and Proteins from Human Blood. Mass Spectrom. Rev. 2011, 30, 685-732.
(7) Rifai, N.; Gillette, M. A.; Carr, S. A. Protein Biomarker Discovery and Validation: The Long and Uncertain Path to Clinical Utility. Nat. Biotechnol. 2006, 24, 971-983.
(8) Kaur, G.; Poljak, A.; Ali, S. A.; Zhong, L.; Raftery, M. J.; Sachdev, P. Extending the Depth of Human Plasma Proteome Coverage Using Simple Fractionation Techniques. J. Proteome Res. 2021, 20, 1261-1279.
(9) Tu, C.; Rudnick, P. A.; Martinez, M. Y.; Cheek, K. L.; Stein, S. E.; Slebos, R. J. C.; Liebler, D. C. Depletion of Abundant Plasma Proteins and Limitations of Plasma Proteomics. J. Proteome Res. 2010, 9, 4982-4991.
(10) Zhao, Y.; Xue, Q.; Wang, M.; Meng, B.; Jiang, Y.; Zhai, R.; Zhang, Y.; Dai, X.; Fang, X. Evolution of Mass Spectrometry Instruments and Techniques for Blood Proteomics. J. Proteome Res. 2023, 22, 1009-1023.
(11) Whiteaker, J. R.; Zhang, H.; Eng, J. K.; Fang, R.; Piening, B. D.; Feng, L.-C.; Lorentzen, T. D.; Schoenherr, R. M.; Keane, J. F.; Holzman, T.; Fitzgibbon, M.; Lin; Zhang, H.; Cooke, K.; Liu, T.; Camp, D. G.; Anderson, L.; Watts, J.; Smith, R. D.; McIntosh, M. W.; Paulovich, A. G. Head-to-Head Comparison of Serum Fractionation Techniques. J. Proteome Res. 2007, 6, 828-836.
(12) Xie, S.; Moya, C.; Bilgin, B.; Jayaraman, A.; Walton, S. P. Emerging Affinity-Based Techniques in Proteomics. Expert Rev. Proteomics 2009, 6, 573-583.
(13) Cao, X.; Sandberg, A.; AraUjo, J. E.; Cvetkovski, F.; Berglund, E.; Eriksson, L. E.; Pernemalm, M. Evaluation of Spin Columns for Human Plasma Depletion to Facilitate MS-Based Proteomics Analysis of Plasma. J. Proteome Res. 2021, 20, 4610-4620.
(14) Tognetti, M.; Sklodowski, K.; Müller, S.; Kamber, D.; Muntel, J.; Bruderer, R.; Reiter, L. Biomarker Candidates for Tumors Identified from Deep-Profiled Plasma Stem Predominantly from the Low Abundant Area. J. Proteome Res. 2022, 21, 1718-1735.
(15) Petrič, T. Č.; Brne, P.; Gabor, B.; Govednik, L.; Barut, M.; Strancar, A.; Kralj, L. Z. Anion-Exchange Chromatography Using Short Monolithic Columns as a Complementary Technique for Human Serum Albumin Depletion Prior to Human Plasma Proteome Analysis. J. Pharm. Biomed. Anal. 2007, 43, 243-249.
(16) Marshall, J.; Jankowski, A.; Furesz, S.; Kireeva, I.; Barker, L.; Dombrovsky, M.; Zhu, W.; Jacks, K.; Ingratta, L.; Bruin, J.; Kristensen, E.; Zhang, R.; Stanton, E.; Takahashi, M.; Jackowski, G. Human Serum Proteins Preseparated by Electrophoresis or Chromatography Followed by Tandem Mass Spectrometry. J. Proteome Res. 2004, 3, 364-382.
(17) Rice, S. J.; Belani, C. P. Characterization of Effective, Simple, and Low-Cost Precipitation Methods for Depleting Abundant Plasma Proteins to Enhance the Depth and Breadth of Plasma Proteomics. PROTEOMICS 2024, 24, 2400071.
(18) Viode, A.; van Zalm, P.; Smolen, K. K.; Fatou, B.; Stevenson, D.; Jha, M.; Levy, O.; Steen, J.; Steen, H.; On behalf of the IMPACC Network. A Simple, Time- and Cost-Effective, High-Throughput Depletion Strategy for Deep Plasma Proteomics. Sci. Adv. 2023, 9, eadf9717.
(19) Liu, G.; Zhao, Y.; Angeles, A.; Hamuro, L. L.; Arnold, M. E.; Shen, J. X. A Novel and Cost Effective Method of Removing Excess Albumin from Plasma/Serum Samples and Its Impacts on LC-MS/MS Bioanalysis of Therapeutic Proteins. Anal. Chem. 2014, 86, 8336-8343.
(20) Blume, J. E.; Manning, W. C.; Troiano, G.; Homburg, D.; Figa, M.; Hesterberg, L.; Platt, T. L.; Zhao, X.; Cuaresma, R. A.; Everley, P. A.; Ko, M.; Liou, H.; Mahoney, M.; Ferdosi, S.; Elgierari, E. M.; Stolarczyk, C.; Tangeysh, B.; Xia, H.; Benz, R.; Siddiqui, A.; et al., Rapid, Deep and Precise Profiling of the Plasma Proteome with Multi-Nanoparticle Protein Corona. Nat. Commun. 2020, 11, 3662.
(21) Suhre, K.; Venkataraman, G. R.; Guturu, H.; Halama, A.; Stephan, N.; Thareja, G.; Sarwath, H.; Motamedchaboki, K.; Donovan, M. K. R.; Siddiqui, A.; Batzoglou, S.; Schmidt, F. Nanoparticle Enrichment Mass-Spectrometry Proteomics Identifies Protein-Altering Variants for Precise pQTL Mapping. Nat. Commun. 2024, 15, 989.
(22) Huang, C.-F.; Hollas, M. A.; Sanchez, A.; Bhattacharya, M.; Ho, G.; Sundaresan, A.; Caldwell, M. A.; Zhao, X.; Benz, R.; Siddiqui, A.; Kelleher, N. L. Deep Profiling of Plasma Proteoforms with Engineered Nanoparticles for Top-Down Proteomics. J. Proteome Res. 2024, 23, 4694-4703.
(23) Thulasiraman, V.; Lin, S.; Gheorghiu, L.; Lathrop, J.; Lomas, L.; Hammond, D.; Boschetti, E. Reduction of the Concentration Difference of Proteins in Biological Liquids Using a Library of Combinatorial Ligands. ELECTROPHORESIS 2005, 26, 3561-3571.
(24) Guerrier, L.; Thulasiraman, V.; Castagna, A.; Fortis, F.; Lin, S.; Lomas, L.; Righetti, P. G.; Boschetti, E. Reducing Protein Concentration Range of Biological Samples Using Solid-Phase Ligand Libraries☆. J. Chromatogr. B 2006, 833, 33-40.
(25) Boschetti, E.; Righetti, P. G. The ProteoMiner in the Proteomic Arena: A Non-Depleting Tool for Discovering Low-Abundance Species. J. Proteomics 2008, 71, 255-264.
(26) Murphy, S.; Dowling, P. DIGE Analysis of ProteoMiner™ Fractionated Serum/Plasma Samples. In Difference Gel Electrophoresis: Methods and Protocols; Ohlendieck, K., Ed.; Springer US: New York, NY, 2023; pp 119-125.
(27) Shen, Y.; Kim, J.; Strittmatter, E. F.; Jacobs, J. M.; Camp II, D. G.; Fang, R.; Tolié, N.; Moore, R. J.; Smith, R. D. Characterization of the Human Blood Plasma Proteome. PROTEOMICS 2005, 5, 4034-4045. https://doi.org/10.1002/pmic.200401246.
(28) Gianazza, E.; Miller, I.; Palazzolo, L.; Parravicini, C.; Eberini, I. With or without You—Proteomics with or without Major Plasma/Serum Proteins. J. Proteomics 2016, 140, 62-80.
(29) Ignjatovic, V.; Geyer, P. E.; Palaniappan, K. K.; Chaaban, J. E.; Omenn, G. S.; Baker, M. S.; Deutsch, E. W.; Schwenk, J. M. Mass Spectrometry-Based Plasma Proteomics: Considerations from Sample Collection to Achieving Translational Data. J. Proteome Res. 2019, 18, 4085-4097.
(30) Abbatiello, S. E.; Schilling, B.; Mani, D. R.; Zimmerman, L. J.; Hall, S. C.; MacLean, B.; Albertolle, M.; Allen, S.; Burgess, M.; Cusack, M. P.; Gosh, M.; Hedrick, V.; Held, J. M.; Inerowicz, H. D.; Jackson, A.; Keshishian, H.; Kinsinger, C. R.; Lyssand, J.; Makowski, L.; Mesri, M.; et al. Large-Scale Interlaboratory Study to Develop, Analytically Validate and Apply Highly Multiplexed, Quantitative Peptide Assays to Measure Cancer-Relevant Proteins in Plasma*. Mol. Cell. Proteomics 2015, 14, 2357-2374.
(31) Li, J.-R.; Sculley, J.; Zhou, H.-C. Metal-Organic Frameworks for Separations. Chem. Rev. 2012, 112, 869-932.
(32) Swinnen, S.; de Azambuja, F.; Parac-Vogt, T. N. From Nanozymes to Multi-Purpose Nanomaterials: The Potential of Metal-Organic Frameworks for Proteomics Applications. Adv. Healthc. Mater. n/a, 2401547.
(33) Hu, Z.; Chen, Z.; Chen, X.; Wang, J. Advances in the Adsorption/Enrichment of Proteins/Peptides by Metal-Organic Frameworks-Affinity Adsorbents. TrAC Trends Anal. Chem. 2022, 153, 116627.
(34) Li, H.; Eddaoudi, M.; O'Keeffe, M.; Yaghi, O. M. Design and Synthesis of an Exceptionally Stable and Highly Porous Metal-Organic Framework. Nature 1999, 402, 276-279.
(35) Peng, J.; Zhang, H.; Li, X.; Liu, S.; Zhao, X.; Wu, J.; Kang, X.; Qin, H.; Pan, Z.; Wu, R. Dual-Metal Centered Zirconium-Organic Framework: A Metal-Affinity Probe for Highly Specific Interaction with Phosphopeptides. ACS Appl. Mater. Interfaces 2016, 8, 35012-35020.
(36) Zhang, Y.-W.; Li, Z.; Zhao, Q.; Zhou, Y.-L.; Liu, H.-W.; Zhang, X.-X. A Facilely Synthesized Amino-Functionalized Metal-Organic Framework for Highly Specific and Efficient Enrichment of Glycopeptides. Chem. Commun. 2014, 50, 11504-11506.
(37) Xie, Y.; Deng, C.; Li, Y. Designed Synthesis of Ultra-Hydrophilic Sulfo-Functionalized Metal-Organic Frameworks with a Magnetic Core for Highly Efficient Enrichment of the N-Linked Glycopeptides. J. Chromatogr. A 2017, 1508, 1-6.
(38) Gu, Z.-Y.; Chen, Y.-J.; Jiang, J.-Q.; Yan, X.-P. Metal-Organic Frameworks for Efficient Enrichment of Peptides with Simultaneous Exclusion of Proteins from Complex Biological Samples. Chem. Commun. 2011, 47, 4787-4789.
(39) Liu, Q.; Sun, N.; Deng, C. Recent Advances in Metal-Organic Frameworks for Separation and Enrichment in Proteomics Analysis. TrAC Trends Anal. Chem. 2019, 110, 66-80.
(40) Cohen, S. M. Postsynthetic Methods for the Functionalization of Metal-Organic Frameworks. Chem. Rev. 2012, 112, 970-1000.
(41) Sha, F.; Xie, H.; Son, F. A.; Kim, K. S.; Gong, W.; Su, S.; Ma, K.; Wang, X.; Wang, X.; Farha, O. K. Rationally Tailored Mesoporous Hosts for Optimal Protein Encapsulation. J. Am. Chem. Soc. 2023, 145, 16383-16390.
(42) Weng, Y.; Chen, R.; Hui, Y.; Chen, D.; Zhao, C.-X. Boosting Enzyme Activity in Enzyme Metal-Organic Framework Composites. Chem Bio Eng. 2024, 1, 99-112.
(43) Liang, W.; Wied, P.; Carraro, F.; Sumby, C. J.; Nidetzky, B.; Tsung, C.-K.; Falcaro, P.; Doonan, C. J. Metal-Organic Framework-Based Enzyme Biocomposites. Chem. Rev. 2021, 121, 1077-1129.
(44) Chen, Y.; Li, P.; Noh, H.; Kung, C.-W.; Buru, C. T.; Wang, X.; Zhang, X.; Farha, O. K. Stabilization of Formate Dehydrogenase in a Metal-Organic Framework for Bioelectrocatalytic Reduction of CO2. Angew. Chem. Int. Ed. 2019, 58, 7682-7686.
(45) Chen, Y.; Li, P.; Modica, J. A.; Drout, R. J.; Farha, 0. K. Acid-Resistant Mesoporous Metal-Organic Framework toward Oral Insulin Delivery: Protein Encapsulation, Protection, and Release. J. Am. Chem. Soc. 2018, 140, 5678-5681.
(46) Chen, W.-H.; Vazquez-Gonzalez, M.; Zoabi, A.; Abu-Reziq, R.; Willner, I. Biocatalytic Cascades Driven by Enzymes Encapsulated in Metal-Organic Framework Nanoparticles. Nat. Catal. 2018, 1, 689-695.
(47) Lian, X.; Fang, Y.; Joseph, E.; Wang, Q.; Li, J.; Banerjee, S.; Lollar, C.; Wang, X.; Zhou, H.-C. Enzyme-MOF (Metal-Organic Framework) Composites. Chem. Soc. Rev. 2017, 46, 3386-3401.
(48) Lykourinou, V.; Chen, Y.; Wang, X.-S.; Meng, L.; Hoang, T.; Ming, L.-J.; Musselman, R. L.; Ma, S. Immobilization of MP-11 into a Mesoporous Metal-Organic Framework, MP-11@mesoMOF: A New Platform for Enzymatic Catalysis. J. Am. Chem. Soc. 2011, 133, 10382-10385.
(49) Chen, Y.; Jimenez-Angeles, F.; Qiao, B.; Krzyaniak, M. D.; Sha, F.; Kato, S.; Gong, X.; Buru, C. T.; Chen, Z.; Zhang, X.; Gianneschi, N. C.; Wasielewski, M. R.; Olvera de la Cruz, M.; Farha, O. K. Insights into the Enhanced Catalytic Activity of Cytochrome c When Encapsulated in a Metal-Organic Framework. J. Am. Chem. Soc. 2020, 142, 18576-18582.
(50) Shieh, F.-K.; Wang, S.-C.; Yen, C.-.; Wu, C.-C.; Dutta, S.; Chou, L.-Y.; Morabito, J. V.; Hu, P.; Hsu, M.-H.; Wu, K. C.-W.; Tsung, C.-K. Imparting Functionality to Biocatalysts via Embedding Enzymes into Nanoporous Materials by a de Novo Approach: Size-Selective Sheltering of Catalase in Metal-Organic Framework Microcrystals. J. Am. Chem. Soc. 2015, 137, 4276-4279.
(51) Wu, X.; Yue, H.; Zhang, Y.; Gao, X.; Li, X.; Wang, L.; Cao, Y.; Hou, M.; An, H.; Zhang, L.; Li, S.; Ma, J.; Lin, H.; Fu, Y.; Gu, H.; Lou, W.; Wei, W.; Zare, R. N.; Ge, J. Packaging and Delivering Enzymes by Amorphous Metal-Organic Frameworks. Nat. Commun. 2019, 10, 5165.
(52) Liang, W.; Xu, H.; Carraro, F.; Maddigan, N. K.; Li, Q.; Bell, S. G.; Huang, D. M.; Tarzia, A.; Solomon, M. B.; Amenitsch, H.; Vaccari, L.; Sumby, C. J.; Falcaro, P.; Doonan, C. J. Enhanced Activity of Enzymes Encapsulated in Hydrophilic Metal—Organic Frameworks. J. Am. Chem. Soc. 2019, 141, 2348-2355.
(53) Lian, X.; Chen, Y.-P.; Liu, T.-F.; Zhou, H.-C. Coupling Two Enzymes into a Tandem Nanoreactor Utilizing a Hierarchically Structured MOF. Chem. Sci. 2016, 7, 6969-6973.
(54) Huang, S.; Kou, X.; Shen, J.; Chen, G.; Ouyang, G. “Armor-Plating” Enzymes with Metal-Organic Frameworks (MOFs). Angew. Chem. Int. Ed. 2020, 59, 8786-8798.
(55) Liang, K.; Ricco, R.; Doherty, C. M.; Styles, M. J.; Bell, S.; Kirby, N.; Mudie, S.; Haylock, D.; Hill, A. J.; Doonan, C. J.; Falcaro, P. Biomimetic Mineralization of Metal-Organic Frameworks as Protective Coatings for Biomacromolecules. Nat. Commun. 2015, 6, 7240.
(56) Lyu, F.; Zhang, Y.; Zare, R. N.; Ge, J.; Liu, Z. One-Pot Synthesis of Protein-Embedded Metal-Organic Frameworks with Enhanced Biological Activities. Nano Lett. 2014, 14, 5761-5765.
(57) Liu, Y.; Cui, S.; Ma, W.; Wu, Y.; Xin, R.; Bai, Y.; Chen, Z.; Xu, J.; Ge, J. Direct Imaging of Protein Clusters in Metal-Organic Frameworks. J. Am. Chem. Soc. 2024, 146, 12565-12576.
(58) Murty, R.; Bera, M. K.; Walton, I. M.; Whetzel, C.; Prausnitz, M. R.; Walton, K. S. Interrogating Encapsulated Protein Structure within Metal-Organic Frameworks at Elevated Temperature. J. Am. Chem. Soc. 2023, 145, 7323-7330.
(59) Cases Diaz, J.; Lozano-Torres, B.; Gimenez-Marques, M. Boosting Protein Encapsulation through Lewis-Acid-Mediated Metal-Organic Framework Mineralization: Toward Effective Intracellular Delivery. Chem. Mater. 2022, 34, 7817-7827.
(60) Huang, A.; Tong, L.; Kou, X.; Gao, R.; Li, Z.-W.; Huang, S.; Zhu, F.; Chen, G.; Ouyang, G. Structural and Functional Insights into the Biomineralized Zeolite Imidazole Frameworks. ACS Nano 2023, 17, 24130-24140.
(61) Park, K. S.; Ni, Z.; Cöté, A. P.; Choi, J. Y.; Huang, R.; Uribe-Romo, F. J.; Chae, H. K.; O'Keeffe, M.; Yaghi, O. M. Exceptional Chemical and Thermal Stability of Zeolitic Imidazolate Frameworks. Proc. Natl. Acad. Sci. 2006, 103, 10186-10191.
(62) Zou, D.; Yu, L.; Sun, Q.; Hui, Y.; Tengjisi; Liu, Y.; Yang, G.; Wibowo, D.; Zhao, C.-X. A General Approach for Biomimetic Mineralization of MOF Particles Using Biomolecules. Colloids Surf. B Biointerfaces 2020, 193, 111108.
(63) Maddigan, N. K.; Tarzia, A.; Huang, D. M.; Sumby, C. J.; Bell, S. G.; Falcaro, P.; Doonan, C. J. Protein Surface Functionalisation as a General Strategy for Facilitating Biomimetic Mineralisation of ZIF-8. Chem. Sci. 2018, 9, 4217-4223.
(64) Zheng, Q.; Sheng, J.; Liu, J.; Chen, X.; Wang, M. Histidine-Rich Protein Accelerates the Biomineralization of Zeolitic Imidazolate Frameworks for In Vivo Protein Delivery. Biomacromolecules 2023, 24, 5132-5141.
(65) Jiang, Y.; Rex, D. A. B.; Schuster, D.; Neely, B. A.; Rosano, G. L.; Volkmar, N.; Momenzadeh, A.; Peters-Clarke, T. M.; Egbert, S. B.; Kreimer, S.; Doud, E. H.; Crook, O. M.; Yadav, A. K.; Vanuopadath, M.; Hegeman, A. D.; Mayta, M. L.; Duboff, A. G.; Riley, N. M.; Moritz, R. L.; Meyer, J. G. Comprehensive Overview of Bottom-Up Proteomics Using Mass Spectrometry. ACS Meas. Sci. Au 2024, 4, 338-417.
(66) Aebersold, R.; Mann, M. Mass-Spectrometric Exploration of Proteome Structure and Function. Nature 2016, 537, 347-355. https://doi.org/10.1038/nature19949.
(67) Zhang, Y.; Fonslow, B. R.; Shan, B.; Baek, M.-C.; Yates, J. R. I. Protein Analysis by Shotgun/Bottom-up Proteomics. Chem. Rev. 2013, 113, 2343-2394.
(68) Roberts, D. S.; Loo, J. A.; Tsybin, Y. O.; Liu, X.; Wu, S.; Chamot-Rooke, J.; Agar, J. N.; Pasa-Tolid, L.; Smith, L. M.; Ge, Y. Top-down Proteomics. Nat. Rev. Methods Primer2024, 4, 1-23.
(69) Toby, T. K.; Fornelli, L.; Kelleher, N. L. Progress in Top-Down Proteomics and the Analysis of Proteoforms. Annu. Rev. Anal. Chem. 2016, 9, 499-519.
(70) Carpenter, B. P.; Talosig, A. R.; Mulvey, J. T.; Merham, J. G.; Esquivel, J.; Rose, B.; Ogata, A. F.; Fishman, D. A.; Patterson, J. P. Role of Molecular Modification and Protein Folding in the Nucleation and Growth of Protein-Metal-Organic Frameworks. Chem. Mater. 2022, 34, 8336-8344.
(71) Liang, W.; Ricco, R.; Maddigan, N. K.; Dickinson, R. P.; Xu, H.; Li, Q.; Sumby, C. J.; Bell, S. G.; Falcaro, P.; Doonan, C. J. Control of Structure Topology and Spatial Distribution of Biomacromolecules in Protein@ZIF-8 Biocomposites. Chem. Mater. 2018, 30, 1069-1077.
(72) Wu, C.; Xie, D.; Mei, Y.; Xiu, Z.; Poduska, K. M.; Li, D.; Xu, B.; Sun, D. Unveiling the Thermolysis Natures of ZIF-8 and ZIF-67 by Employing in Situ Structural Characterization Studies. Phys. Chem. Chem. Phys. 2019, 21, 17571-17577.
(73) Hu, Y.; Kazemian, H.; Rohani, S.; Huang, Y.; Song, Y. In Situ High Pressure Study of ZIF-8 by FTIR Spectroscopy. Chem. Commun. 2011, 47, 12694-12696.
(74) Gao, J.; Chu, W.; Ding, X.; Ding, L.; Guo, Q.; Fu, Y. Degradation Kinetic Studies of BSA@ZIF-8 Nanoparticles with Various Zinc Precursors, Metal-to-Ligand Ratios, and pH Conditions. ACS Omega 2023, 8, 44601-44610.
(75) Akimbekov, Z.; Katsenis, A. D.; Nagabhushana, G. P.; Ayoub, G.; Arhangelskis, M.; Morris, A. J.; Friscid, T.; Navrotsky, A. Experimental and Theoretical Evaluation of the Stability of True MOF Polymorphs Explains Their Mechanochemical Interconversions. J. Am. Chem. Soc. 2017, 139, 7952-7957.
(76) Wang, C.; Sudlow, G.; Wang, Z.; Cao, S.; Jiang, Q.; Neiner, A.; Morrissey, J. J.; Kharasch, E. D.; Achilefu, S.; Singamaneni, S. Metal-Organic Framework Encapsulation Preserves the Bioactivity of Protein Therapeutics. Adv. Healthc. Mater. 2018, 7, 1800950.
(77) Brown, K. A.; Chen, B.; Guardado-Alvarez, T. M.; Lin, Z.; Hwang, L.; Ayaz-Guner, S.; Jin, S.; Ge, Y. A Photocleavable Surfactant for Top-down Proteomics. Nat. Methods 2019, 16, 417-420.
(78) Brown, K. A.; Tucholski, T.; Eken, C.; Knott, S.; Zhu, Y.; Jin, S.; Ge, Y. High-Throughput Proteomics Enabled by a Photocleavable Surfactant. Angew. Chem. Int. Ed. 2020, 59, 8406-8410.
(79) Chan, B. A.; Hughes, B. G. M. Targeted Therapy for Non-Small Cell Lung Cancer: Current Standards and the Promise of the Future. Transl. Lung Cancer Res. 2015, 4.
(80) Labrande, C.; Velly, L.; Canolle, B.; Guillet, B.; Masmejean, F.; Nieoullon, A.; Pisano, P. Neuroprotective Effects of Tacrolimus (FK506) in a Model of Ischemic Cortical Cell Cultures: Role of Glutamate Uptake and FK506 Binding Protein 12 kDa. Neuroscience 2006, 137, 231-239.
(81) Khan, A. A.; Alsahli, M. A.; Rahmani, A. H. Myeloperoxidase as an Active Disease Biomarker: Recent Biochemical and Pathological Perspectives. Med. Sci. 2018, 6, 33.
(82) Ramachandra, C. J. A.; Ja, K. P. M. M.; Chua, J.; Cong, S.; Shim, W.; Hausenloy, D. J. Myeloperoxidase As a Multifaceted Target for Cardiovascular Protection. Antioxid. Redox Signal. 2020, 32, 1135-1149.
(83) Lam, C. S. P.; Lund, L. H.; Shah, S. J.; Voors, A. A.; Erlinge, D.; Saraste, A.; Pirazzi, C.; Grove, E. L.; Barasa, A.; Schou, M.; Aziz, A.; Svedlund, S.; Wijngaarden, J. V.; Lindstedt, E.-L.; Gustavsson, A.; Nelander, K.; Garkaviy, P.; Gan, L.-M.; Gabrielsen, A. Myeloperoxidase Inhibition in Heart Failure With Preserved or Mildly Reduced Ejection Fraction: SATELLITE Trial Results. J. Card. Fail. 2024, 30, 104-110.
(84) Lund, L. H.; Lam, C. S. P.; Pizzato, P. E.; Gabrielsen, A.; Michaslsson, E.; Nelander, K.; Ericsson, H.; Holden, J.; Folkvaljon, F.; Mattsson, A.; Collen, A.; Aurell, M.; Whatling, C.; Baldus, S.; Drelich, G.; Goudev, A.; Merkely, B.; Bergh, N.; Shah, S. J. Rationale and Design of ENDEAVOR: A Sequential Phase 2b-3 Randomized Clinical Trial to Evaluate the Effect of Myeloperoxidase Inhibition on Symptoms and Exercise Capacity in Heart Failure with Preserved or Mildly Reduced Ejection Fraction. Eur. J. Heart Fail. 2023, 25, 1696-1707.
(85) Zhang, L.; Smyth, D.; Al-Khalaf, M.; Blet, A.; Du, Q.; Bernick, J.; Gong, M.; Chi, X.; Oh, Y.; Roba-Oshin, M.; Coletta, E.; Feletou, M.; Gramolini, A. O.; Kim, K.-H.; Coutinho, T.; Januzzi, J. L.; Tyl, B.; Ziegler, A.; Liu, P. P. Insulin-like Growth Factor-Binding Protein-7 (IGFBP7) Links Senescence to Heart Failure. Nat. Cardiovasc. Res. 2022, 1, 1195-1214.
(86) Turner, A. J. Chapter 79—Aminopeptidase N. In Handbook of Proteolytic Enzymes (Third Edition); Rawlings, N. D., Salvesen, G., Eds.; Academic Press, 2013; pp 397-403.
(87) Yang, J.; Goetz, D.; Li, J.-Y.; Wang, W.; Mori, K.; Setlik, D.; Du, T.; Erdjument-Bromage, H.; Tempst, P.; Strong, R.; Barasch, J. An Iron Delivery Pathway Mediated by a Lipocalin. Mol. Cell 2002, 10, 1045-1056.
(88) Standker, L.; Schrader, M.; Kanse, S. M.; Jürgens, M.; Forssmann, W.-G.; Preissner, K. T. Isolation and Characterization of the Circulating Form of Human Endostatin. FEBS Lett. 1997, 420, 129-133.
(89) Stoner, A.; Harris, A.; Oddone, F.; Belamkar, A.; Vercellin, A. C. V.; Shin, J.; Januleviciene, I.; Siesky, B. Topical Carbonic Anhydrase Inhibitors and Glaucoma in 2021: Where Do We Stand? Br. J. Ophthalmol. 2022, 106, 1332-1337.
(90) Behl, T.; Chadha, S.; Sehgal, A.; Singh, S.; Sharma, N.; Kaur, R.; Bhatia, S.; Al-Harrasi, A.; Chigurupati, S.; Alhowail, A.; Bungau, S. Exploring the Role of Cathepsin in Rheumatoid Arthritis. Saudi J. Biol. Sci. 2022, 29, 402-410.
(91) Trainor, P. J.; Brambatti, M.; Carlisle, S. M.; Mullick, A. E.; Shah, S. J.; Kahlon, T.; Mostacero, D. O.; Mousavi, H.; Morgan, E. S.; Tami, Y.; Michos, E. D.; Ouyang, P.; Tsimikas, S.; DeFilippis, A. P. Blood Levels of Angiotensinogen and Hypertension in the Multi-Ethnic Study of Atherosclerosis (MESA). J. Am. Coll. Cardiol. 2023, 81, 1248-1259.
(92) Fournier, T.; Medjoubi-N, N.; Porquet, D. Alpha-1-Acid Glycoprotein. Biochim. Biophys. Acta BBA—Protein Struct. Mol. Enzymol. 2000, 1482, 157-171.
(93) Ruiz, M. Into the Labyrinth of the Lipocalin Al-Acid Glycoprotein. Front. Physiol. 2021, 12.
(94) Shen, Y.; Jacobs, J. M.; Camp, D. G.; Fang, R.; Moore, R. J.; Smith, R. D.; Xiao, W.; Davis, R. W.; Tompkins, R. G. Ultra-High-Efficiency Strong Cation Exchange LC/RPLC/MS/MS for High Dynamic Range Characterization of the Human Plasma Proteome. Anal. Chem. 2004, 76, 1134-1144.
(95) Adkins, J. N.; Varnum, S. M.; Auberry, K. J.; Moore, R. J.; Angell, N. H.; Smith, R. D.; Springer, D. L.; Pounds, J. G. Toward a Human Blood Serum Proteome: Analysis By Multidimensional Separation Coupled With Mass Spectrometry*, ¶., Mol. Cell. Proteomics 2002, 1, 947-955.
(96) Verwey, N. A.; Schuitemaker, A.; van der Flier, W. M.; Mulder, S. D.; Mulder, C.; Hack, C. E.; Scheltens, P.; Blankenstein, M. A.; Veerhuis, R. Serum Amyloid P Component as a Biomarker in Mild Cognitive Impairment and Alzheimer's Disease. Dement. Geriatr. Cogn. Disord. 2008, 26, 522-527.
(97) Chapman, E. A.; Roberts, D. S.; Tiambeng, T. N.; Andrews, J.; Wang, M.-D.; Reasoner, E. A.; Melby, J. A.; Li, B. H.; Kim, D.; Alpert, A. J.; Jin, S.; Ge, Y. Structure and Dynamics of Endogenous Cardiac Troponin Complex in Human Heart Tissue Captured by Native Nanoproteomics. Nat. Commun. 2023, 14, 8400.
(98) Tiambeng, T. N.; Roberts, D. S.; Brown, K. A.; Zhu, Y.; Chen, B.; Wu, Z.; Mitchell, S. D.; Guardado-Alvarez, T. M.; Jin, S.; Ge, Y. Nanoproteomics Enables Proteoform-Resolved Analysis of Low-Abundance Proteins in Human Serum. Nat. Commun. 2020, 11, 3903.
(99) Roberts, D. S.; Chen, B.; Tiambeng, T. N.; Wu, Z.; Ge, Y.; Jin, S. Reproducible Large-Scale Synthesis of Surface Silanized Nanoparticles as an Enabling Nanoproteomics Platform: Enrichment of the Human Heart Phosphoproteome. Nano Res. 2019, 12, 1473-1481.
(100) Aballo, T. J.; Roberts, D. S.; Melby, J. A.; Buck, K. M.; Brown, K. A.; Ge, Y. Ultrafast and Reproducible Proteomics from Small Amounts of Heart Tissue Enabled by Azo and timsTOF Pro. J. Proteome Res. 2021, 20, 4203-4211.
(101) Aballo, T. J.; Roberts, D. S.; Bayne, E. F.; Zhu, W.; Walcott, G.; Mahmoud, A. I.; Zhang, J.; Ge, Y. Integrated Proteomics Reveals Alterations in Sarcomere Composition and Developmental Processes during Postnatal Swine Heart Development. J. Mol. Cell. Cardiol. 2023, 176, 33-40.
(102) Buck, K. M.; Roberts, D. S.; Aballo, T. J.; Inman, D. R.; Jin, S.; Ponik, S.; Brown, K. A.; Ge, Y. One-Pot Exosome Proteomics Enabled by a Photocleavable Surfactant. Anal. Chem. 2022, 94, 7164-7168.
(103) Meier, F.; Brunner, A.-D.; Koch, S.; Koch, H.; Lubeck, M.; Krause, M.; Goedecke, N.; Decker, J.; Kosinski, T.; Park, M. A.; Bache, N.; Hoerning, O.; Cox, J.; Rather, O.; Mann, M. Online Parallel Accumulation-Serial Fragmentation (PASEF) with a Novel Trapped Ion Mobility Mass Spectrometer. Mol. Cell. Proteomics 2018, 17, 2534-2545.
(104) Kong, A. T.; Leprevost, F. V.; Avtonomov, D. M.; Mellacheruvu, D.; Nesvizhskii, A. I. MSFragger: Ultrafast and Comprehensive Peptide Identification in Mass Spectrometry-Based Proteomics. Nat. Methods 2017, 14, 513-520.
(105) Wieczorek, S.; Combes, F.; Lazar, C.; Giai Gianetto, Q.; Gatto, L.; Dorffer, A.; Hesse, A.-M.; Coute, Y.; Ferro, M.; Bruley, C.; Burger, T. DAPAR & ProStaR: Software to Perform Statistical Analyses in Quantitative Discovery Proteomics. Bioinformatics 2017, 33, 135-136.
(106) Zhang, X.; Smits, A. H.; van Tilburg, G. B.; Ovaa, H.; Huber, W.; Vermeulen, M. Proteome-Wide Identification of Ubiquitin Interactions Using UbIA-MS. Nat. Protoc. 2018, 13, 530-550.
(107) Tucholski, T.; Cai, W.; Gregorich, Z. R.; Bayne, E. F.; Mitchell, S. D.; Mcllwain, S. J.; de Lange, W. J.; Wrobbel, M.; Karp, H.; Hite, Z.; Vikhorev, P. G.; Marston, S. B.; Lal, S.; Li, A.; dos Remedios, C.; Kohmoto, T.; Hermsen, J.; Ralphe, J. C.; Kamp, T. J.; Moss, R. L.; Ge, Y. Distinct Hypertrophic Cardiomyopathy Genotypes Result in Convergent Sarcomeric Proteoform Profiles Revealed by Top-down Proteomics. Proc. Natl. Acad. Sci. 2020, 117, 24691-24700.
(108) Bayne, E. F.; Rossler, K. J.; Gregorich, Z. R.; Aballo, T. J.; Roberts, D. S.; Chapman, E. A.; Guo, W.; Palecek, S. P.; Ralphe, J. C.; Kamp, T. J.; Ge, Y. Top-down Proteomics of Myosin Light Chain Isoforms Define Chamber-Specific Expression in the Human Heart. J. Mol. Cell. Cardiol. 2023, 181, 89-97.
(109) Chapman, E. A.; Aballo, T. J.; Melby, J. A.; Zhou, T.; Price, S. J.; Rossler, K. J.; Lei, I.; Tang, P. C.; Ge, Y. Defining the Sarcomeric Proteoform Landscape in Ischemic Cardiomyopathy by Top-Down Proteomics. J. Proteome Res. 2023, 22, 931-941.
(110) Larson, E. J.; Pergande, M. R.; Moss, M. E.; Rossler, K. J.; Wenger, R. K.; Krichel, B.; Josyer, H.; Melby, J. A.; Roberts, D. S.; Pike, K.; Shi, Z.; Chan, H.-J.; Knight, B.; Rogers, H. T.; Brown, K. A.; Ong, I. M.; Jeong, K.; Marty, M. T.; Mcllwain, S. J.; Ge, Y. MASH Native: A Unified Solution for Native Top-down Proteomics Data Processing. Bioinformatics 2023, 39, btad359.

1. A method for separating biomolecules in a mixture comprising the steps of: encapsulating one or more biomolecules in the mixture within a metal-organic framework (MOF), thereby forming encapsulated biomolecule@MOF composites, wherein encapsulating the one or more biomolecules comprises adding a) an organic ligand, and b) metal ions or a metal ion precursor able to form the metal ions, to an aqueous solution comprising a biological fluid containing the mixture, wherein the organic ligand and metal ions surround each of the one or more biomolecules and form the encapsulated biomolecule@MOF composites; and separating the encapsulated biomolecule@MOF composites in the aqueous solution from biomolecules that are not encapsulated.

2. The method of claim 1, wherein the one or more biomolecules are proteins or polypeptides.

3. The method of claim 1, wherein the aqueous solution comprises a whole blood sample, blood plasma sample, or blood serum sample, from a subject.

4. The method of claim 1, wherein the aqueous solution comprises a first buffer solution able to maintain a native structure of the biomolecules, and wherein the method further comprises replacing the first buffer solution with a mass spectrometry buffer solution after the encapsulated biomolecule@MOF composites are separated from the aqueous solution, and performing mass spectrometry analysis on the separated unencapsulated biomolecules, encapsulated biomolecule@MOF composites, or both.

5. The method of claim 1 comprising collecting the separated encapsulated biomolecule@MOF composites, degrading the MOF, and recovering the one or more biomolecules.

6. The method of claim 1, wherein the organic ligand has the formula: 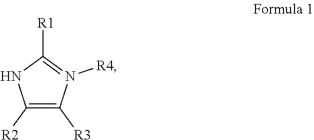
wherein R1, R2, and R3, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group, and
R4 is absent or is selected from the group consisting of a phosphate group, sulphate group, and C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group, wherein if R4 is present, the double bond between the adjacent nitrogen atom and the neighboring carbon atom will be reduced to a single bond,
wherein, optionally, any two of R1-R4 together form a heterocyclic group, aryl groups, or heteroaryl group.

7. The method of claim 6, wherein R1, R2, and R3, individually from each other, are selected from the group consisting of a hydrogen and C₁-C₂ alkyl groups and R4 is absent.

8. The method of claim 1, wherein the organic ligand is 2-methylimidazole (2-Melm) and the metal ion precursor is zinc acetate dihydrate.

9. The method of claim 1, wherein the organic ligand has the formula: 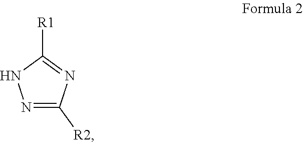
wherein R1 and R2, individually from each other, are selected from the group consisting of a hydrogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group.

10. The method of claim 9, wherein R1 and R2, individually from each other, are selected from the group consisting of a hydrogen and C₁-C₄ alkyl groups, wherein one or more of the C₁-C₄ alkyl groups are optionally haloalkyl groups comprising one or more halogen atoms.

11. The method of claim 1, wherein the organic ligand has the formula

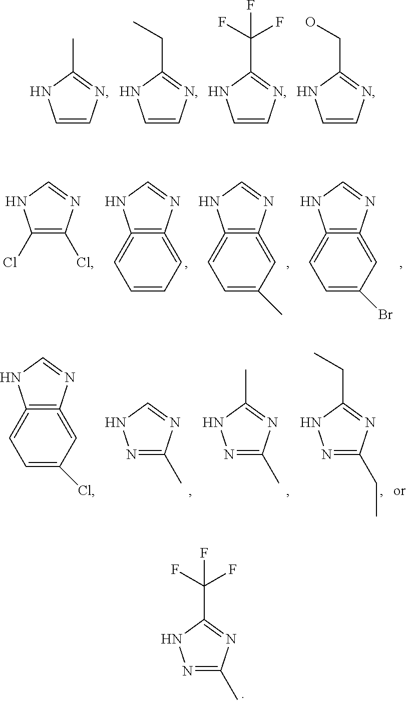

12. The method of claim 1, wherein the organic ligand has the formula 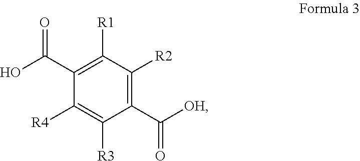
wherein R1, R2, R3 and R4, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group.

13. The method of claim 1, wherein the organic ligand has the formula

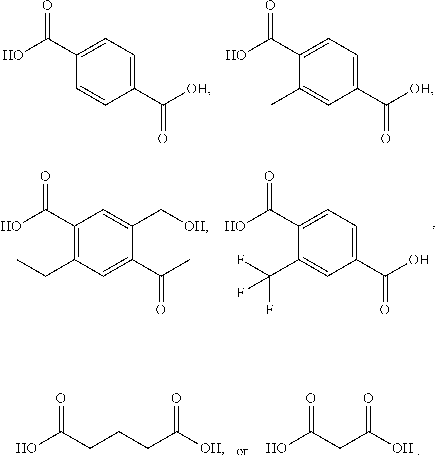

14. The method of claim 1, wherein the metal ions are selected from the group consisting of zinc ions, iron ions, cobalt ions, copper ions, zirconium ions, aluminum ions, nickel ions, calcium ions, and combinations thereof.

15. The method of claim 1, comprising adding a) a second organic ligand and b) second metal ions or a second metal ion precursor able to form the second metal ions, to the aqueous solution after the encapsulated biomolecule@MOF composites are separated,
wherein the second organic ligand and second metal ions surround one or more additional biomolecules and form one or more additional encapsulated biomolecule@MOF composites,
wherein the second organic ligand is different from the first organic ligand, and the second metal ions are the same or different than the first metal ions, and
wherein the one or more additional encapsulated biomolecule@MOF composites are separated from remaining biomolecules in the aqueous solution that are not encapsulated.

16. A method for analyzing proteins in a blood sample comprising the steps of:
encapsulating one or more proteins in the blood sample within a metal-organic framework (MOF), thereby forming encapsulated protein@MOF composites, wherein encapsulating the one or more proteins comprises adding a) an organic ligand, and b) metal ions or a metal ion precursor able to form the metal ions, to the blood sample, wherein the organic ligand and metal ions surround each of the one or more proteins and form the encapsulated protein@MOF composites;
separating the encapsulated protein@MOF composites from proteins that are not encapsulated, thereby generating a blood sample fraction that does not contain the one or more proteins; and
performing one or more analytical techniques on the blood sample fraction that does not contain the one or more proteins.

17. The method of claim 16, wherein the one or more proteins that are encapsulated and separated comprise one or more proteins selected from the group consisting of:
albumin, complement C3, serotransferrin, alpha-2-macroglobulin, alpha-1-antitrypsin, apolipoprotein A-I, apolipoprotein A-II, haptoglobin, immunoglobulin heavy constant gamma 1, immunoglobulin heavy constant gamma 2, immunoglobulin heavy constant gamma 3, immunoglobulin heavy constant gamma 4, immunoglobulin heavy constant alpha 1, immunoglobulin heavy constant alpha 2, immunoglobulin heavy constant mu, transthyretin, fibrinogen alpha chain, fibrinogen beta chain, and fibrinogen gamma chain.

18. The method of claim 16, wherein at least 90% of the one or more proteins are removed from the blood sample.

19. The method of claim 16 wherein the organic ligand has the formula: 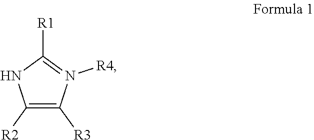
wherein R1, R2, and R3, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group, and
R4 is absent or is selected from the group consisting of a phosphate group, sulphate group, and C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group, wherein if R4 is present, the double bond between the adjacent nitrogen atom and the neighboring carbon atom will be reduced to a single bond,
wherein, optionally, any two of R1-R4 together form a heterocyclic group, aryl groups, or heteroaryl group.

20. The method of claim 19, wherein R1, R2, and R3, individually from each other, are selected from the group consisting of a hydrogen and C₁-C₂ alkyl groups and R4 is absent.

21. The method of claim 16, wherein the organic ligand has the formula: 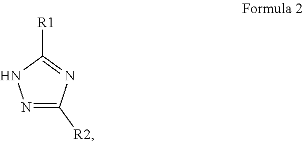
wherein R1 and R2, individually from each other, are selected from the group consisting of a hydrogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group.

22. The method of claim 21, wherein R1 and R2, individually from each other, are selected from the group consisting of a hydrogen and C₁-C₄ alkyl groups, wherein one or more of the C₁-C₄ alkyl groups are optionally haloalkyl groups comprising one or more halogen atoms.

23. The method of claim 16, wherein the organic ligand has the formula 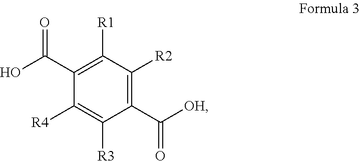
wherein R1, R2, R3 and R4, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group.

24. The method of claim 16, wherein the metal ions are selected from the group consisting of zinc ions, iron ions, cobalt ions, copper ions, zirconium ions, aluminum ions, nickel ions, calcium ions, and combinations thereof.

25. The method of claim 16, wherein encapsulating the one or more proteins comprises mixing the organic ligand and metal ions with the one or proteins for 5 minutes to 4 hours at a temperature between 18° C. and 25° C.

26. A kit for separating proteins in an aqueous solution, said kit comprising:
a first metal-organic framework (MOF) reagent comprising a first organic ligand, a first buffer solution, and a first metal ion precursor able to form first metal ions in the aqueous solution, wherein the first organic ligand and first metal ions are able to selectively form a first encapsulated protein@MOF composite comprising one or more proteins in the aqueous solution; and
a second MOF reagent comprising a second organic ligand, a second buffer solution, and a second metal ion precursor able to form second metal ions in the aqueous solution, wherein the second organic ligand and second metal ions are able to selectively form a second encapsulated protein@MOF composite around one or more different proteins in the aqueous solution,
wherein the second organic ligand is different from the first organic ligand, and the second metal ions are the same or different than the first metal ions,
wherein the first and second buffer solutions have a pH between 5.0 and 10.0; and
wherein the first and second organic ligands are each present in an amount so as to generate a concentration between 0.05 M to 3.0 M when added to the aqueous solution, and the first and second ion precursors are each present in an amount so as to generate a concentration between 0.01 M to 2.0 M when added to the aqueous solution.

27. The kit of claim 26, wherein one of the first and second organic ligand has the formula: 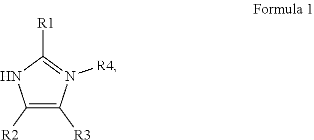
wherein R1, R2, and R3, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group, and
R4 is absent or is selected from the group consisting of a phosphate group, sulphate group, and C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group, wherein if R4 is present, the double bond between the adjacent nitrogen atom and the neighboring carbon atom will be reduced to a single bond,
wherein, optionally, any two of R1-R4 together form a heterocyclic group, aryl groups, or heteroaryl group.

28. The kit of claim 26, wherein one of the first and second organic ligand has the formula: 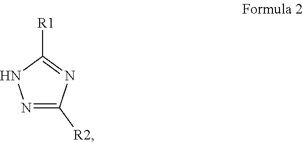
wherein R1 and R2, individually from each other, are selected from the group consisting of a hydrogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group.

29. The kit of claim 26, wherein one of the first and second organic ligand has the formula: 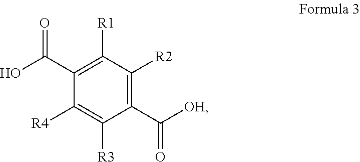
wherein R1, R2, R3 and R4, individually from each other, are selected from the group consisting of a hydrogen, phosphate group, sulphate group, nitro group, halogen, C₁-C₁₂ alkyl group, C₂-C₁₂ alkenyl group, C₁-C₁₂ alkoxy group, C₁-C₁₂ aldehyde group, C₁-C₁₂ ether group, C₁-C₁₂ ester group, and C₁-C₁₂ amino group.

30. The kit of claim 26, wherein the first metal ion precursor and the first organic ligand are present in an amount so as to generate between a 1:2 and 1:5 molar ratio when added to the aqueous solution, and wherein the second metal ion precursor and the second organic ligand are present in an amount so as to generate between a 1:2 and 1:5 molar ratio when added to the aqueous solution.
